(19) United States
(12) Patent Application Publication
(10) Pub. No.: US 2025/0025418 A1
(43) Pub. Date: Jan. 23, 2025

(54) HYALURONIC ACID-LIPID DERIVATIVE, LIPID NANOPARTICLE COMPRISING SAME, AND USE THEREOF

(71) Applicants: GENEXINE, INC., Seoul (KR); PHI BIOMED INC., Seoul (KR); POSTECH RESEARCH AND BUSINESS DEVELOPMENT FOUNDATION, Pohang-si, Gyeongsangbuk-do-do (KR)

(72) Inventors: Sei Kwang HAHN, Pohang-si (KR); Young Chul SUNG, Seoul (KR); Daham JEONG, Seoul (KR); Mun Gu KIM, Pohang-si (KR); Hwan Hee KIM, Seoul (KR)

(73) Assignees: GENEXINE, INC., Seoul (KR); PHI BIOMED INC., Seoul (KR); POSTECH RESEARCH AND BUSINESS DEVELOPMENT FOUNDATION, Pohang-si, Gyeongsangbuk-do-do (KR)

(21) Appl. No.: 18/577,456

(22) PCT Filed: Jul. 7, 2022
(86) PCT No.: PCT/KR2022/009827

§ 371 Date: Sep. 23, 2024

(30) Foreign Application Priority Data
Jul. 7, 2021 (KR) 10-2021-0088956
Jul. 9, 2021 (KR) 10-2021-0090563
Jul. 12, 2021 (KR) 10-2021-0090679

(51) Int. Cl. A61K 9/127; A61K 9/51; A61K 39/00; A61K 39/39

(52) U.S. Cl. CPC A61K 9/1272; A61K 9/5123; A61K 9/5161; A61K 39/39; A61K 2039/53; A61K 2039/55555

(57) ABSTRACT

A hyaluronic acid-lipid derivative, lipid nanoparticles comprising the same, and uses thereof are disclosed. The lipid nanoparticles comprising the hyaluronic acid-lipid derivative can be utilized as a vaccine or a drug delivery carrier for effectively carrying mRNA, proteins and other drugs, and stably delivering same into the body, and such a vaccine or drug delivery carrier has excellent mucoadhesive properties and mucosal permeability and thus has the advantage of enabling vaccine or drug delivery via the nasal cavity and lungs.

### TECHNICAL FIELD

The present invention relates to a hyaluronic acid-lipid derivative, a lipid nanoparticle including the same and use thereof, and more specifically to a hyaluronic acid-lipid derivative in which a lipid having a hydrophobic alkyl chain is bonded to low-molecular-weight hyaluronic acid, a lipid nanoparticle including the same and use thereof.

### BACKGROUND ART

Messenger RNA is a nucleic acid material that expresses its own protein according to the central dogma of molecular biology, and currently, Pfizer and Moderna's COVID-19 vaccines (Cominati, RNA-1273), which have been officially approved by the FDA, use an mRNA vaccine platform. These vaccines express the spike protein, which is a part of the SARS-COV-2 virus, from the genetic information stored in mRNA, and the body's immune cells recognize the same and induce an immune response. In terms of expressing the protein, Moderna and Pfizer's vaccines do not use the spike protein mRNA sequence of SARS-COV-2 as it is, but are characterized in that they have appropriately changed the mRNA sequence for the purpose of stabilizing the protein structure and facilitating recognition as an antigen (e.g., proline substitution (K986P, V987P) mutations) (Dai, L., Gao. G. F., Viral targets for vaccines against COVID-19, Nat Rev Immunol, 2021).

However, mRNA vaccines have chronic problems such as excessively high immunogenicity, low in vivo stability and low protein expression. For example, mRNA has very low stability, unlike DNA-based vaccines, and it is difficult to transport and store because it contains a lot of water in its formulation or is easily deformed in a room temperature environment.

Meanwhile, mRNA-lipid nanoparticle (mRNA-LNP) is a complex for effectively delivering mRNA encoding a target antigen into cells, and unlike viral vector-based vaccines, it does not form an immunogen for carriers, and unlike DNA vaccines, there is no possibility of being integrated into the genome, and it has the advantage of showing a high immunogen expression rate.

The LNP which is used to effectively load and deliver mRNA into cells is composed of 4 major lipids, and specifically, there are 1) a cationic lipid which is capable of forming an electrostatic complex with mRNA, 2) phosphatidylcholine, which helps cellular uptake of the lipid bilayer and promotes destabilization after uptake, 3) PEGylated lipids for hindering the fusion and aggregation of LNPs, and 4) cholesterol which contributes to the structure and fusibility of LNPs. Since the mRNA loading efficacy and intracellular delivery efficacy of LNPs vary depending on the type of these major lipids, the development of LNPs is essential for the development of an effective mRNA vaccine delivery system.

As a complex for effectively delivering mRNA into cells, Moderna discloses an mRNA formulation including a fusogenic lipid, cholesterol and PEG lipid, and specifically, it discloses that the formulation may have a molar ratio of 50:10:38.5:1.5 to 3.0 (cationic lipid:fusogenic lipid:cholesterol:PEG lipid) (Korean Patent Application Laid-Open No. 10-2014-0102759), and for example, it uses SM-102. DMG-PEG, cholesterol and DSPC, which are generally used for nucleic acid delivery.

Meanwhile, in the case of Pfizer and BioNTech, ALC-0315 and ALC-0159, which have developed by themselves, are used together with cholesterol and DSPC as a complex for effectively delivering mRNA into cells (European Patent Publication No. 3,901,261).

However, in the case of PEGylated lipids, anti-PEG antibodies can be generated in patients, and as a result, there is a problem of reduced vaccine efficacy due to accelerated blood clearance (ABC), which accelerates the removal of an LNP agent administered by binding specifically to the PEG part, and thus, the immunogenicity of the PEG molecule must be reduced or the alternative routes of administration must be considered. In addition, in the case of PEGylated lipids, they have the effect of preventing the aggregation of LNP and blood flow, but since the cellular uptake rate is low, the development of new helper lipids is necessary.

In order to improve these problems, the inventors of the present invention have made intensive efforts to develop a new LNP platform technique for mRNA vaccine delivery that can overcome the limitations of PEGylated lipids, and as a result, when the lipid nanoparticle including a hyaluronic acid-lipid derivative is used, it was confirmed that the mRNA can be effectively loaded in the vaccine and delivered into the body, and it has excellent mucoadhesion and pulmonary delivery performance while maintaining stability in the body, thereby completing the present invention.

### Disclosure

### Technical Problem

An object of the present invention is to provide a hyaluronic acid-lipid derivative having improved mucoadhesion and mucosal permeability, a lipid nanoparticle including the hyaluronic acid-lipid derivative, and a vaccine composition including the lipid nanoparticle.

### Technical Solution

In order to achieve the above object, the present invention provides a lipid nanoparticle, including a hyaluronic acid-lipid derivative.

In the present invention, the lipid nanoparticle may further include an ionic lipid, cholesterol and phosphatidylcholine.

In the present invention, the ionic lipid may be a cationic lipid.

In the present invention, the hyaluronic acid-lipid derivative may be formed by introducing a lipid into the —COOH group or —OH group of a hyaluronic acid sugar skeleton or a reducing sugar at the terminal of hyaluronic acid.

In the present invention, in the hyaluronic acid-lipid derivative, the lipid may be a lipid having a hydrophobic alkyl chain.

In the present invention, the hyaluronic acid-lipid derivative may be a hyaluronic acid-cholesterol derivative or hyaluronic acid-1,2-dimyristoyl-rac-glycerol. In the present invention, the lipid nanoparticle may further include lapidated PEG.

For example, the lapidated PEG may be DMG-PEG, but the present invention is not limited thereto.

In the present invention, the lipid nanoparticle may be for delivering a nucleic acid, a protein or a drug.

In the present invention, the nucleic acid may be mRNA.

In the present invention, the molecular weight of the hyaluronic acid may be 3,000 to 6,000 Da.

In the present invention, the molar ratio of ionic lipid:cholesterol:phosphatidylcholine:hyaluronic acid-lipid derivative in the lipid nanoparticle may be 30 to 60:30 to 60:8 to 12:1 to 10.

In the present invention, the lipid nanoparticle may be administered by a route selected from the group consisting of intravenous, intramuscular, intravitreal, intrathecal, intratumoral, intranasal, pulmonary and subcutaneous routes.

In addition, the present invention provides a vaccine composition, including an mRNA vaccine and the lipid nanoparticle.

In addition, the present invention relates to a hyaluronic acid-lipid derivative, which is represented by any one of Chemical Formulas 1 to 4 below:

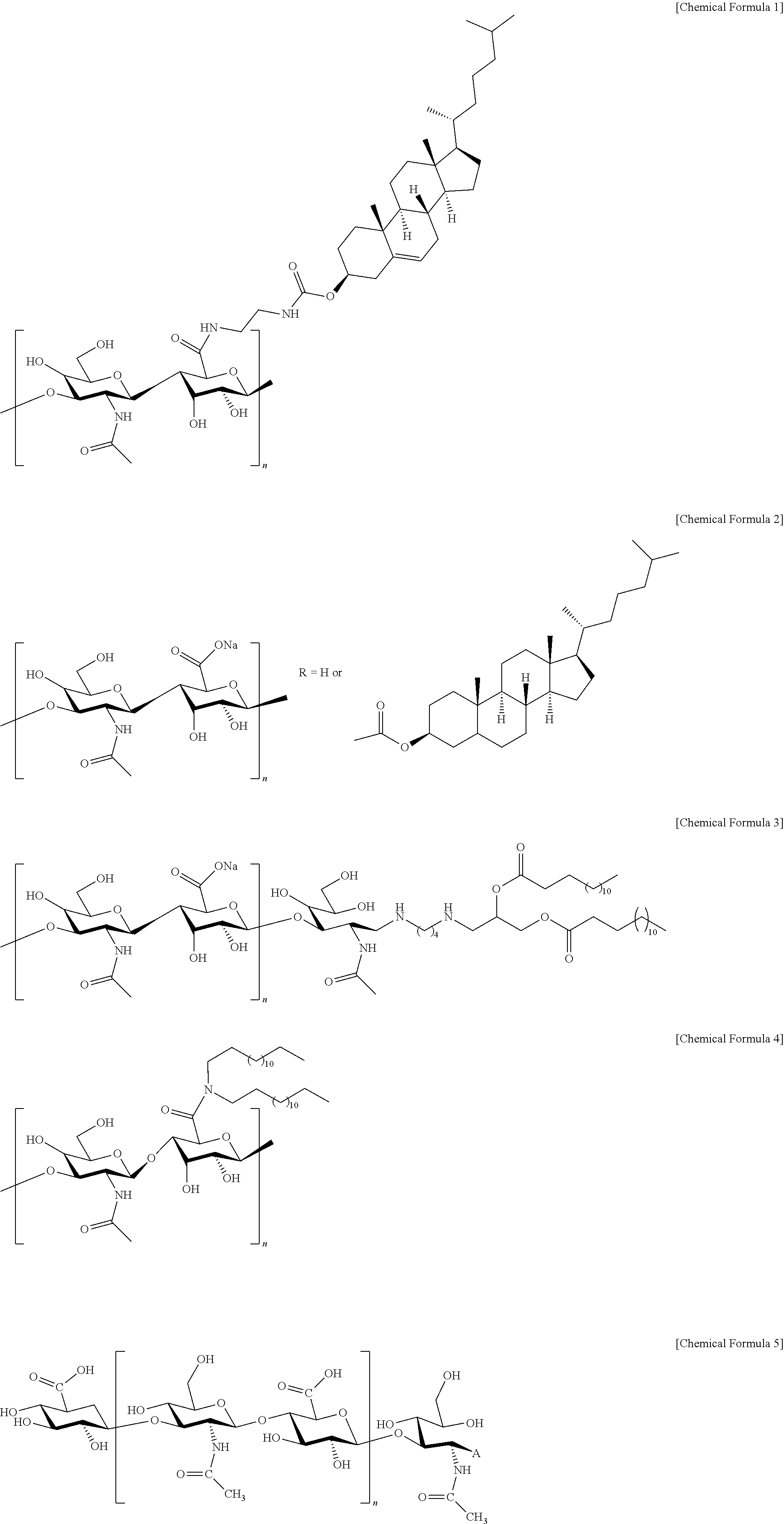

wherein in Chemical Formulas 1 to 5, n is an integer from 1 to 50, and in Chemical Formula 5, A is an aldehyde group.

In the present invention, the compound of Chemical Formula 1 or Chemical Formula 2 may be prepared by substituting the —OH group of hyaluronic acid with cholesteryl chloroformate without chemical modification.

In the present invention, the hyaluronic acid-lipid derivative may control the degree of cholesterol substitution of the hyaluronic acid-cholesterol derivative by adjusting the equivalent amounts of cholesteryl chloroformate, 4-dimethylaminopyridine (DMAP) and triethylamine (TEA).

In the present invention, the compound of Chemical Formula 3 may be prepared by substituting the terminal of a hyaluronic acid reducing sugar with 1,2-dimyristoyl-rac-glycerol.

In the present invention, the terminal of the hyaluronic acid reducing sugar may be substituted with an amine group through a ring-opening reaction.

In the present invention, the amine group may be substituted by using a diamine selected from the group consisting of ethylenediamine, butylenediamine, hexamethylenediamine, pentaethylenehexamine, 1,4-butanediamine and 1,5-diamino-2-methylpentane.

### Advantageous Effects

The lipid nanoparticle including a hyaluronic acid-lipid derivative according to the present invention can be utilized as a vaccine or a drug carrier that effectively loads mRNA, protein and other drugs, and stably delivers the same to the body, and since such a vaccine or drug carrier has excellent mucoadhesion and mucosal permeability, it has the advantage of being able to deliver the vaccine or drug through the nasal cavity and lungs.

### DESCRIPTION OF DRAWINGS

FIG. 1 is a synthesis diagram showing the synthesis process of hyaluronic acid cholesterol derivative 1 (HA-CHOL).

FIG. 2 is a synthesis diagram showing the synthesis process of hyaluronic acid cholesterol derivative 2 (CHA).

FIG. 3a is the results of 1H NMR spectrum analysis of hyaluronic acid-cholesterol derivative 1.

FIG. 3b is the results of 1H NMR spectrum analysis of various hyaluronic acid-cholesterol derivatives 2.

FIG. 4 is a synthesis diagram showing the synthesis process of hyaluronic acid-1,2-Dimyristoyl-rac-glycerol derivative (DMG-HA).

FIG. 5 is the results of 1H NMR spectrum analysis of HA-b-damb.

FIG. 6 is the results of 1H NMR spectrum analysis of HA-b-damb-TBA.

FIG. 7 is the results of 1H NMR spectrum analysis of Tosyl-DMG.

FIG. 8 is the results of 1H NMR spectrum analysis of DMG-HA. (a) NMR solvent D₂O, (b) NMR solvent DMSO-d6.

FIG. 9 shows a process diagram for preparing LNPs including hyaluronic acid-lipid derivatives.

FIG. 10 is a comparison of the sizes of LNPs prepared in a microfluidic mixing device and in vitro. (a) Size dispersion of LNPs prepared from DMG-PEG/SM-102/DSPC/cholesterol, composed with a molar ratio of 1:50:10:39. (b) Size dispersion of LNPs prepared with 5K CHA/SM-102/DSPC/cholesterol (m=prepared by microfluidic mixing device, p=prepared by in vitro mixing), composed with a molar ratio of 1:50:10:39.

FIG. 11 shows LNP size dispersion according to the content of 5K CHA.

FIG. 12 shows LNP size dispersion according to the content of DMG-HA.

FIG. 13 shows the size distribution of LNPs including DMG-PEG, DMG-PEG/4K CHA and 4K CHA, respectively.

FIG. 14 shows the zeta potential of LNPs including DMG-PEG/4K CHA, 4K CHA, DMG-HA and DMG-PEG, respectively.

FIG. 15 shows the size distribution of LNPs including a PEG derivative and a hyaluronic acid-lipid derivative together.

FIG. 16 shows the zeta potential of LNPs including a PEG derivative and a hyaluronic acid-lipid derivative together.

FIG. 17 shows the results of confirming GFP expression by carrying GFP mRNA in LNPs including DMG-PEG/4K CHA, DMG-HA and DMG-PEG, and delivering the same into A549 cells.

FIG. 18 is a result of quantitative analysis of GFP expression by loading GFP mRNA in LNPs containing both a PEG derivative and a hyaluronic acid-lipid derivative and delivering them into A549 cells. (a) DMG-PEG/4K CHA, (b) DMG-PEG/DMG-HA, (c) DMG-PEG, (d) Lipofectamine

FIG. 19 is the results of confirming the loading efficiency by Ribogreen assay by loading mRNA on LNPs by varying the content of 5k CHA or including DMG-PEG.

FIG. 20 is the results of confirming the loading efficiency by Ribogreen assay after mRNA was loaded on the LNP including a PEG derivative and a hyaluronic acid-lipid derivative together.

### MODES OF THE INVENTION

Unless defined otherwise, all technical and scientific terms used herein have the same meanings as commonly understood by one of ordinary skill in the art to which the present invention pertains. In general, the nomenclature used herein and the experimental methods described below are those that are well-known and commonly used in the art.

In the present invention, as a biopolymer, a hyaluronic acid-lipid derivative that binds a lipid having a hydrophobic alkyl chain to hyaluronic acid, which has excellent biodegradability and biocompatibility, has been developed, and when it is prepared into a lipid nanoparticle, it was confirmed that it can be utilized as a delivery vehicle for drugs including nucleic acids, proteins and other compounds, as well as mRNA.

Hyaluronic acid is a linear biopolymer in which D-glucuronic acid and N-acetyl-D-glucosamine are repeated. Since hyaluronic acid has excellent biocompatibility, it has already been widely used in ophthalmic surgical aids, joint function improving agents, drug delivery materials, eye drops, hydrogel fillers, wrinkle improving agents, cosmetics and the like, and thus, the safety thereof has been proven.

Accordingly, in one aspect, the present invention relates to a lipid nanoparticle, including a hyaluronic acid-lipid derivative.

In the present invention, the lipid nanoparticle may be characterized in that it further includes ionic lipid, cholesterol and phosphatidylcholine, but the present invention is not limited thereto.

In the present invention, the ionic lipid may be characterized in that it is a cationic lipid, but the present invention is not limited thereto.

In the present invention, the cationic lipid refers to a lipid exhibiting a cationic property at neutral pH, and preferably, SM-102 may be used. However, in the present invention, the cationic lipid may be selected from cationic lipids described in PCT Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333, and US Patent Application Publication Nos. US20100036115 and US20120202871, but the present invention is not limited thereto, and each of the above documents is incorporated herein by reference in the present specification. In another exemplary embodiment, the cationic lipid may be selected from Chemical Formula A (but the present invention is not limited thereto) described in PCT Publication Nos. WO2012040184, WO2011153120, WO2011149733, WO2011090965, WO2011043913, WO2011022460, WO2012061259, WO2012054365 and WO2012044638, and each of the above documents is incorporated herein by reference in the present specification. In still another exemplary embodiment, the cationic lipid may be selected from Chemical Formula CLI-CLXXIX of PCT Publication No. WO2008103276, Chemical Formula CLI-CLXXIX of U.S. Pat. No. 7,893,302, Chemical Formula CLI-CLXXXXII of U.S. Pat. No. 7,404,969, Chemical Formula CLI-CLXXXX of US Patent Publication No. US20100036115 and Chemical Formula I-VI of US Patent Application Publication No. US20100036115, but the present invention is not limited thereto, and each of the above documents is incorporated herein by reference in the present specification. By way of non-limiting example, the cationic lipid may be selected from (20Z,23Z)-N,N-dimethylnonacosa-20,23-dien-10-amine, (17Z,20Z)-N,N-dimethylhexacosa-17,20-dien-9-amine, (1Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18X)-N,N-dimethyltetracosa-15,18-dien-5-amine, (14Z,17Z)-N,N-dimethyltricosa-14,17-dien-4-amine, (19Z,22Z)-N,N-dimethyloctacosaoctacosa-19,22-dien-9-amine, (18Z,21Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-diene-10-amine, (21Z,24Z)-N,N-dimethyltriaconta-21,24-dien-9-amine, (18Z)-N,N-dimethylheptacose-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacose-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacose-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]heneicosan-10-amine, N,N-dimethyl-1-[(1S,2S)-2-{[(1R,2R)-2-cyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-1-[(1S,2R)-2-decylcyclopropyl]-N,N-dimethyloctadecane-9-amine, dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy) propan-2-amine, S—N,N-dimethyl-1-[(9Z,12Z)-octadeca-9,12-dien-1-yloxy]-3-(octyloxy) propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-diene-1-yloxy]propan-2-amine, N,N-dimethyl-1-[(9Z)-octadec-9-en-1-yloxy]-3-(octyloxy) propan-2-amine; (2S)—N,N-dimethyl-1-[(6Z,9Z,12Z)-octadeca-6,9,12-trien-1-yloxy]-3-(octyloxy) propan-2-amine, (2S)-1-[(11Z,14Z)-icosa-11,14-dien-1-yloxy]-N, N-dimethyl-3-(pentyloxy) propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy) propan-2-amine, 1-[(13Z,16Z)-docosa-13,16-dien-1-yloxy]-N,N-dimethyl-3-(octyloxy) propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N, N-dimethyl-3-(octyloxy) propan-2-amine, 1-[(9Z)-hexadec-9-en-1-yloxy]-N,N-dimethyl-3-(octyloxy) propan-2-amine, (2R)—N,N-dimethyl-H (1-methoyloctylmethoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy) propan-2-amine, N,N-dimethyl-1-{[8-(2-octylcyclopropyl) octyl]oxy}-3-(octyloxy) propan-2-amine and (11E,20Z,23Z)-N,N-dimethylnonacosa-11,20,2-trien-10-amine, or a pharmaceutically acceptable salt thereof or a stereoisomer thereof.

In one aspect, the cationic lipid may be synthesized by the methods that are known in the art and/or described in PCT Publication Nos. WO2012040184, WO2011153120, WO2011149733, WO2011090965, WO2011043913, WO2011022460, WO2012061259, WO2012054365, WO2012044638, WO2010080724 and WO201021865; and each of the above documents is incorporated herein by reference in the present specification.

In the present invention, the cholesterol includes all of cholesterol derivatives having a cholesterol structure, and it may be characterized in that it is included at 30 to 60 mol % based on the total number of moles of molecules constituting the nanoparticles, but the present invention is not limited thereto.

In the present invention, phosphatidylcholine includes all of lipids having a phosphatidylcholine structure, and for example, DSPC (1,2-distearoyl-sn-glycero-3-phosphocholine) may be used. In the present invention, the phosphatidylcholine may be characterized in that it is included in 8 to 12 mol % based on the total number of moles of molecules constituting the nanoparticles, but the present invention is not limited thereto.

In the present invention, the hyaluronic acid-lipid derivative is a general term for compounds in which a lipid is bound to hyaluronic acid, and in the hyaluronic acid-lipid derivative, the lipid may preferably be a lipid having a hydrophobic alkyl chain, but the present invention not limited thereto. The lipid may be a lipid having an alkyl chain with, for example, cholesterol, dimyristroyl glycerol, and N,N ditetradecylacetamide.

In the present invention, the hyaluronic acid-lipid derivative may be characterized in that it is included in 1 to 10 mol % based on the total number of moles of molecules constituting the nanoparticles, but the present invention is not limited thereto.

In the present invention, the hyaluronic acid-lipid derivative may be formed by introducing a lipid into the —COOH group or —OH group of a hyaluronic acid sugar backbone or a reducing sugar at the terminal of hyaluronic acid, but the present invention is not limited thereto.

In the present invention, the hyaluronic acid-lipid derivative may be a hyaluronic acid-cholesterol derivative or hyaluronic acid-1,2-dimyristoyl-rac-glycerol, but the present invention is not limited thereto.

In the present invention, the hyaluronic acid-lipid derivative may be characterized by being represented by any one of Chemical Formulas 1 to 5 below, but the present invention is not limited thereto.

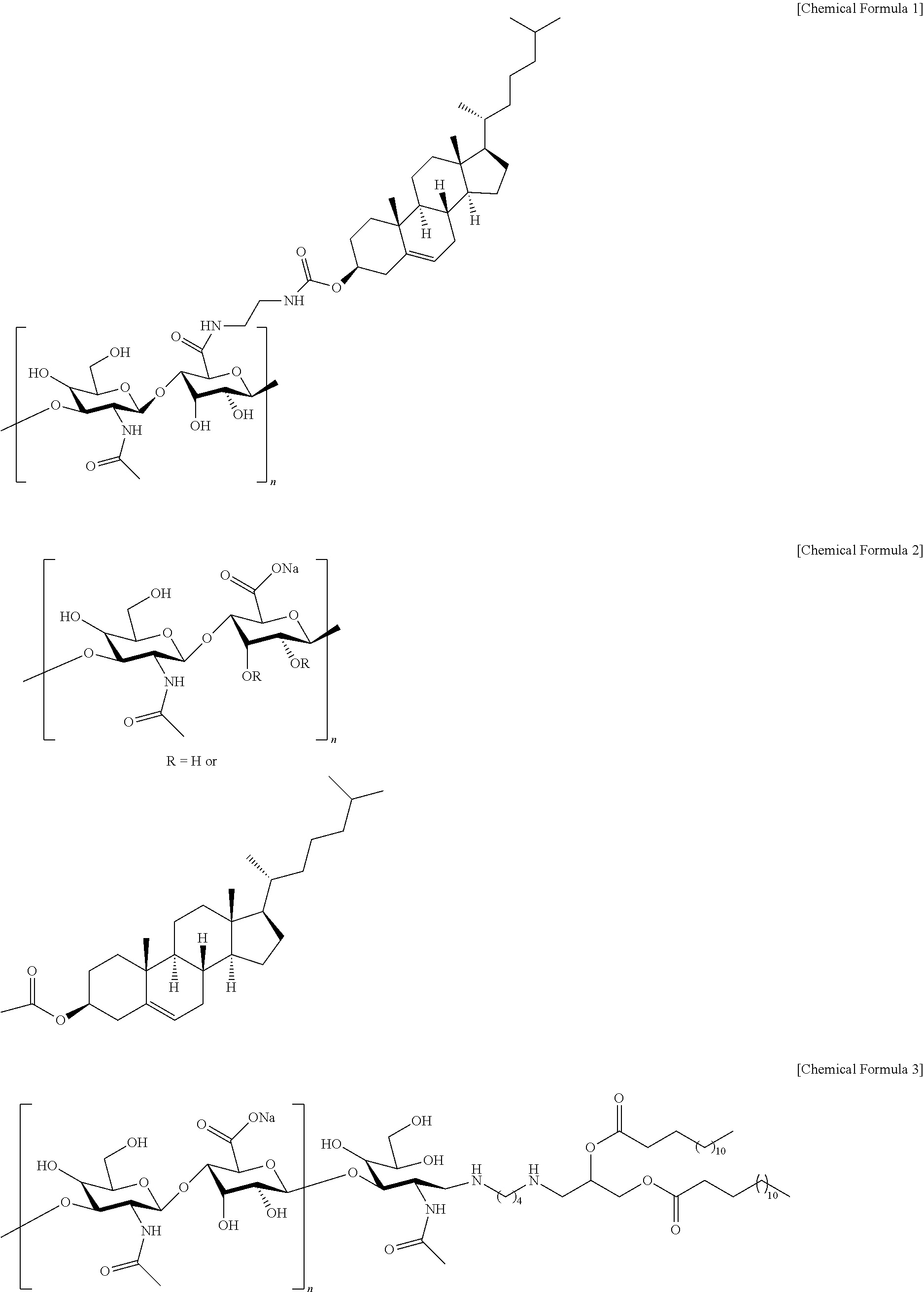

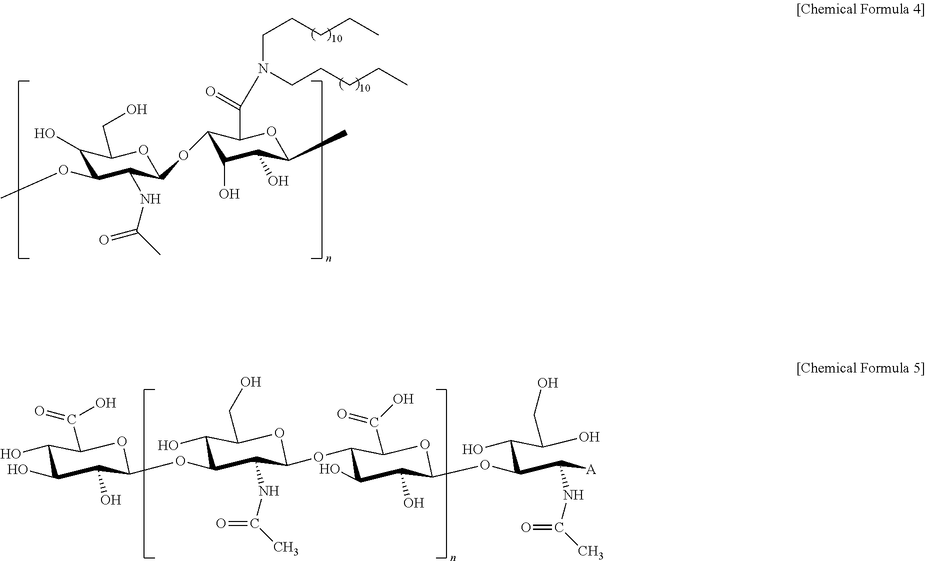

In Chemical Formulas 1 to 5, n is an integer of 1 to 50, and in Chemical Formula 5, A is an aldehyde group.

For example, n may be an integer of 1, 2, 3, 4, 5, 6, 7, 8, 9 or 10.

In the present invention, the lipid nanoparticle may be characterized in that it further includes lipidated PEG. For example, in the present invention, the lipid nanoparticle may further include a PEG derivative.

In the present invention, the lipidated PEG or PEG derivative, such as DMG-PEG, may be characterized in that it is included in 0.5 to 2 mol % based on the total number of moles of molecules constituting the nanoparticles, but the present invention is not limited thereto.

In this case, the mol % of the hyaluronic acid-lipid derivative in the lipid nanoparticles may be reduced. For example, the hyaluronic acid-lipid derivative in the lipid nanoparticles may be included at 0.01 to 10 mol %, and specifically, 0.1 to 10 mol %, but the present invention is not limited thereto.

In the present invention, the lipid nanoparticle may be characterized in that it is for nucleic acid, protein or drug delivery, but the present invention is not limited thereto.

In the present invention, the nucleic acid may be DNA, RNA or PNA, and preferably, mRNA.

In the present invention, the protein may be an antibody in one aspect and a peptide in another aspect.

In the present invention, the drug may be a compound.

Therefore, in the present invention, the lipid nanoparticles may be loaded with target-specific antibodies, proteins, fluorescent dyes and the like depending on the disease.

That is, from another aspect, the present invention relates to the use of a lipid nanoparticle for the delivery of a nucleic acid, protein or drug. In addition, the present invention may provide the use of a lipid nanoparticle in the preparation of pharmaceuticals for nucleic acid, protein or drug delivery.

In the present invention, the molecular weight of the hyaluronic acid may be characterized in that it is 1,000 to 10,000 Da, and preferably, 3,000 to 6,000 Da. For example, the hyaluronic acid may have a molecular weight of about 4,000 Da or about 5,000 Da, but the present invention is not limited thereto.

In the present invention, the molar ratio of the ionic lipid:cholesterol:phosphatidylcholine:hyaluronic acid-lipid derivative in the lipid nanoparticle may be 30 to 60:30 to 60:8 to 12:1 to 10, but the present invention is not limited thereto.

Preferably, the molar ratio of the ionic lipid:cholesterol phosphatidylcholine:hyaluronic acid-lipid derivative in the lipid nanoparticle may be 40 to 50:40 to 50:8 to 12:1 to 10.

In still another preferred aspect, the molar ratio of the ionic lipid:cholesterol:phosphatidylcholine:hyaluronic acid-lipid derivative in the lipid nanoparticle may be 40 to 50:40 to 50:10:1 to 10.

In still another preferred aspect, the molar ratio of the ionic lipid:cholesterol:phosphatidylcholine:hyaluronic acid-lipid derivative in the lipid nanoparticle may be 50:40:10:0.1.

In one aspect, in the lipid nanoparticle, the ionic lipid in the lipid nanoparticle is SM-102, the phosphatidylcholine is DSPC, the hyaluronic acid-lipid derivative is a compound represented by Chemical Formula 2 (4k CHA), and the SM-102:cholesterol:DSPC:4k CHA:DMG-PEG may be included at a molar ratio of 49:39:10:1:1.

In another aspect, in the lipid nanoparticle, the ionic lipid in the lipid nanoparticle is SM-102, the phosphatidylcholine is DSPC, the hyaluronic acid-lipid derivative is a compound represented by Chemical Formula 2 (4k CHA), and the SM-102:cholesterol:DSPC: 4k CHA may be included at a molar ratio of 50:39:10:1.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 3 (DMG-HA), and the SM-102:cholesterol:DSPC:DMG-HA may be included at a molar ratio of 48:38:9:5.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 3 (DMG-HA), and the SM-102:cholesterol:DSPC:DMG-HA may be included at a molar ratio of 50:39:10:1.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 3 (DMG-HA), and the SM-102:cholesterol:DSPC:DMG-HA may be included at a molar ratio of 48:37:10:5.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 3 (DMG-HA), and the SM-102:cholesterol:DSPC:DMG-HA may be included at a molar ratio of 46:35:9:10.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 2 (5k CHA), and the SM-102:cholesterol:DSPC: 5k CHA may be included at a molar ratio of 46:35:9:10.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 2 (5k CHA), and the SM-102:cholesterol:DSPC: 5k CHA may be included at a molar ratio of 47:37:9:7.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 2 (5k CHA), and the SM-102:cholesterol:DSPC: 5k CHA may be included at a molar ratio of 48:38:10:4.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 2 (5k CHA), and the SM-102:cholesterol:DSPC: 5k CHA may be included at a molar ratio of 49:38:10:3.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 3 (DMG-HA), and the SM-102:cholesterol:DSPC:DMG-HA may be included at a molar ratio of 50:39:10:1.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 3 (DMG-HA), and the SM-102:cholesterol:DSPC:DMG-HA may be included at a molar ratio of 48:37:10:5.

In still another aspect, in the lipid nanoparticle, the ionic lipid in the lipid nanoparticle is SM-102, the phosphatidylcholine is DSPC, the hyaluronic acid-lipid derivative is a compound represented by Chemical Formula 3 (DMG-HA), and the SM-102:cholesterol:DSPC:DMG-HA may be included at a molar ratio of 46:35:9:10.

In the present invention, in the lipid nanoparticle, the ionic lipid in the lipid nanoparticle is SM-102, the phosphatidylcholine is DSPC, the hyaluronic acid-lipid derivative is a compound represented by Chemical Formula 2 (CHA), and the SM-102:cholesterol:DSPC:CHA may be included at a molar ratio of 48 to 49:37 to 39:10:2 to 5.

In the present invention, in the lipid nanoparticle, the ionic lipid in the lipid nanoparticle is SM-102, the phosphatidylcholine is DSPC, the hyaluronic acid-lipid derivative is a compound represented by Chemical Formula 2 (CHA), and the SM-102:cholesterol:DSPC:CHA may be included at a molar ratio of 48 to 49:38:10:3 to 4.

In the present invention, in the lipid nanoparticle, the ionic lipid in the lipid nanoparticle is SM-102, the phosphatidylcholine is DSPC, the hyaluronic acid-lipid derivative is a compound represented by Chemical Formula 2 (CHA), and the SM-102:cholesterol:DSPC:CHA may be included at a molar ratio of 47.5 to 48.5:38:10:3.5 to 4.5.

In the present invention, the molar ratio of the ionic lipid:cholesterol:phosphatidylcholine:PEG derivative:hyaluronic acid-lipid derivative in the lipid nanoparticle may be 49 to 51:38 to 40:9 to 11:0.5 to 1.5:0.05 to 0.15.

In a preferred aspect, the molar ratio of the ionic lipid:cholesterol:phosphatidylcholine:DMG-PEG:hyaluronic acid-lipid derivative in the lipid nanoparticle may be 49 to 51:38 to 40:9 to 11:0.5 to 1.5:0.05 to 0.15.

In another preferred aspect, the molar ratio of the ionic lipid:cholesterol:phosphatidylcholine:DMG-PEG:hyaluronic acid-lipid derivative in the lipid nanoparticle may be 50:40:10:1:0.1.

In the present invention, in the lipid nanoparticle, the ionic lipid in the lipid nanoparticle is SM-102, the phosphatidylcholine is DSPC, the hyaluronic acid-lipid derivative is a compound represented by Chemical Formula 2 (CHA), and the SM-102:cholesterol:DSPC:DMG-PEG:CHA may be included at a molar ratio of 49 to 50:39 to 40:10:0.1 to 1:0.1 to 1.

In the present invention, in the lipid nanoparticle, the ionic lipid in the lipid nanoparticle is SM-102, the phosphatidylcholine is DSPC, the hyaluronic acid-lipid derivative is a compound represented by Chemical Formula 3 (DMG-HA), and the SM-102:cholesterol:DSPC:DMG-PEG:DMG-HA may be included at a molar ratio of 49 to 50:39 to 40:10:0.1 to 1:0.1 to 1.

In the present invention, the lipid nanoparticle may be administered by a route selected from the group consisting of intravenous, intramuscular, intravitreal, intrathecal, intratumoral, intranasal, pulmonary and subcutaneous routes, but the present invention is not limited thereto.

In the present invention, the lipid nanoparticles may be prepared by mixing with a buffer solution at a volume ratio of 1:1 to 5, preferably, 1:2 to 4, and more preferably, about 1:3, but the present invention is not limited thereto.

In this case, the buffer may be selected from the group consisting of saline, phosphate buffered saline and Ringers lactate, but the present invention is not limited thereto.

In the present invention, the lipid nanoparticle may be prepared by hydrating the ionic lipid, cholesterol, phosphatidylcholine and hyaluronic acid-lipid derivative with a buffer including nucleic acid (e.g., mRNA), protein or other drug.

In the present invention, the lipid nanoparticle dissolves the ionic lipid, cholesterol, phosphatidylcholine and hyaluronic acid-lipid derivative in an organic solvent which is capable of hydration, and afterwards, a buffer including a nucleic acid (e.g., mRNA), protein or other drug is prepared, and after mixing this solution using a filtration membrane, filter, mixer or stirrer, it may be prepared by removing the ethanol.

If necessary, a step for sterilization or storage may be added during the preparation of the lipid nanoparticle.

In the present invention, the lipid nanoparticle may be mixed with a microfluidic mixing device or in vitro pipetting.

In the present invention, the lipid nanoparticle may be formulated to be administered intranasally, and in this case, it may be formulated in a powder form to minimize water content.

For example, in the case of a powder formulation, it is possible to increase potential local and systemic immune responses by prolonging the retention time in the nasal mucosa by preparing solid lipid nanoparticles through a spray dryer.

In the present invention, particularly, in terms of carrying mRNA, it has the advantage of improving the loading efficiency compared to conventional lipid nanoparticles.

Therefore, in another aspect, the present invention relates to a vaccine composition, including an mRNA vaccine and the lipid nanoparticle.

In the present invention, the vaccine composition may be administered by a route selected from the group consisting of intravenous, intramuscular, intravitreal, intrathecal, intratumoral, intranasal, pulmonary and subcutaneous routes, but the present invention is limited thereto.

The vaccine composition according to the present invention may be formulated in a dosage form as described in the present specification, such as other topical, intranasal, intratracheal or injectable dosage form (e.g., intravenous, intraocular, intravitreal, intramuscular, intracardiac, intraperitoneal or subcutaneous).

Liquid dosage forms for parenteral administration may include pharmaceutically acceptable emulsions, microemulsions, solutions, suspensions, syrups and/or elixirs, but the present invention is not limited thereto. In addition to the active ingredient, liquid dosage forms may include water or other solvents, solubilizers and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (particularly, cottonseed oil, peanut oil, corn oil, germ oil, olive oil, castor oil and sesame oil), glycerol, tetrahydrofurfuryl alcohol, polyethylene glycol and fatty acid ester sorbitan or sorbita, and a mixture thereof, but it may include an inert diluent that is commonly used in the art, including but not limited to the above. In certain exemplary embodiments for parenteral administration, the composition may be mixed with a solubilizing agent such as cremophor, alcohols, oils, modified oils, glycols, polysorbates, cyclodextrins, polymers and/or combinations thereof.

Injectable dosage forms may be sterilized, for example, by filtration through a bacteria-retaining filter and/or by incorporating a sterilizing agent in the form of a sterile solid composition which can be dissolved or dispersed in sterile water or other sterile injectable medium prior to use.

If it is desired to slow the absorption of the active ingredient from subcutaneous or intramuscular injection, in order to prolong the effect of the active ingredient, this may be accomplished by the use of a liquid suspension of a poorly water-soluble crystalline or amorphous substance.

In one aspect, the vaccine composition of the present invention is preferred for pulmonary delivery.

The vaccine composition according to the present invention which is useful for pulmonary delivery may also be used for intranasal delivery. Another dosage form which is suitable for intranasal administration is a powder including the active ingredient and having an average particle size of about 0.2 μm to 500 μm. This dosage form is administered in a nasal manner, that is, by rapid inhalation through the nasal passages from a container of powder that is held close to the nose.

Dosage forms that are suitable for nasal administration may include, for example, from about 0.1% (w/w) to about 100% (w/w) of the active ingredient, and may include one or more additional ingredients described in the present specification.

In the present invention, the vaccine composition may further contain various adjuvants. The adjuvant may be an immune enhancer such as salts that create a pH environment and cytokines that are capable of inducing an immune response, but the present invention is not limited thereto.

Meanwhile, in the vaccine composition according to the present invention, the hyaluronic acid itself may be an immune adjuvant. Low-molecular-weight hyaluronic acid stimulates an immune response to induce the maturation of dendritic cells and the secretion of cytokines, thereby maximizing the effectiveness of vaccines.

In particular, the lipid nanoparticle according to the present invention may have a size of 100 to 1,000 nm, and preferably, 100 to 300 nm, and it may have improved size dispersion compared to conventional lipid nanoparticles.

As such, the small and uniform lipid nanoparticles according to the present invention have advantageous properties for vaccine delivery.

In still another aspect, the present invention relates to the use of a composition including an mRNA vaccine and the lipid nanoparticle in the prevention or treatment of an infectious disease.

In still another aspect, the present invention relates to the use of a composition including an mRNA vaccine and the lipid nanoparticle in the preparation of pharmaceuticals (or drugs) for preventing or treating an infectious disease.

In still another aspect, the present invention relates to a method for preventing or treating an infectious disease, including the step of administering a composition including the mRNA vaccine and the lipid nanoparticle to a subject in need thereof.

Additionally, in the present invention, a hyaluronic acid-lipid derivative having improved biocompatibility, mucoadhesion and mucosal permeability compared to existing lipid nanoparticles was prepared by combining hyaluronic acid with excellent biodegradability and biocompatibility with a lipid.

Accordingly, in still another aspect, the present invention relates to a hyaluronic acid-lipid derivative represented by any one of Chemical Formulas 1 to 5 below:

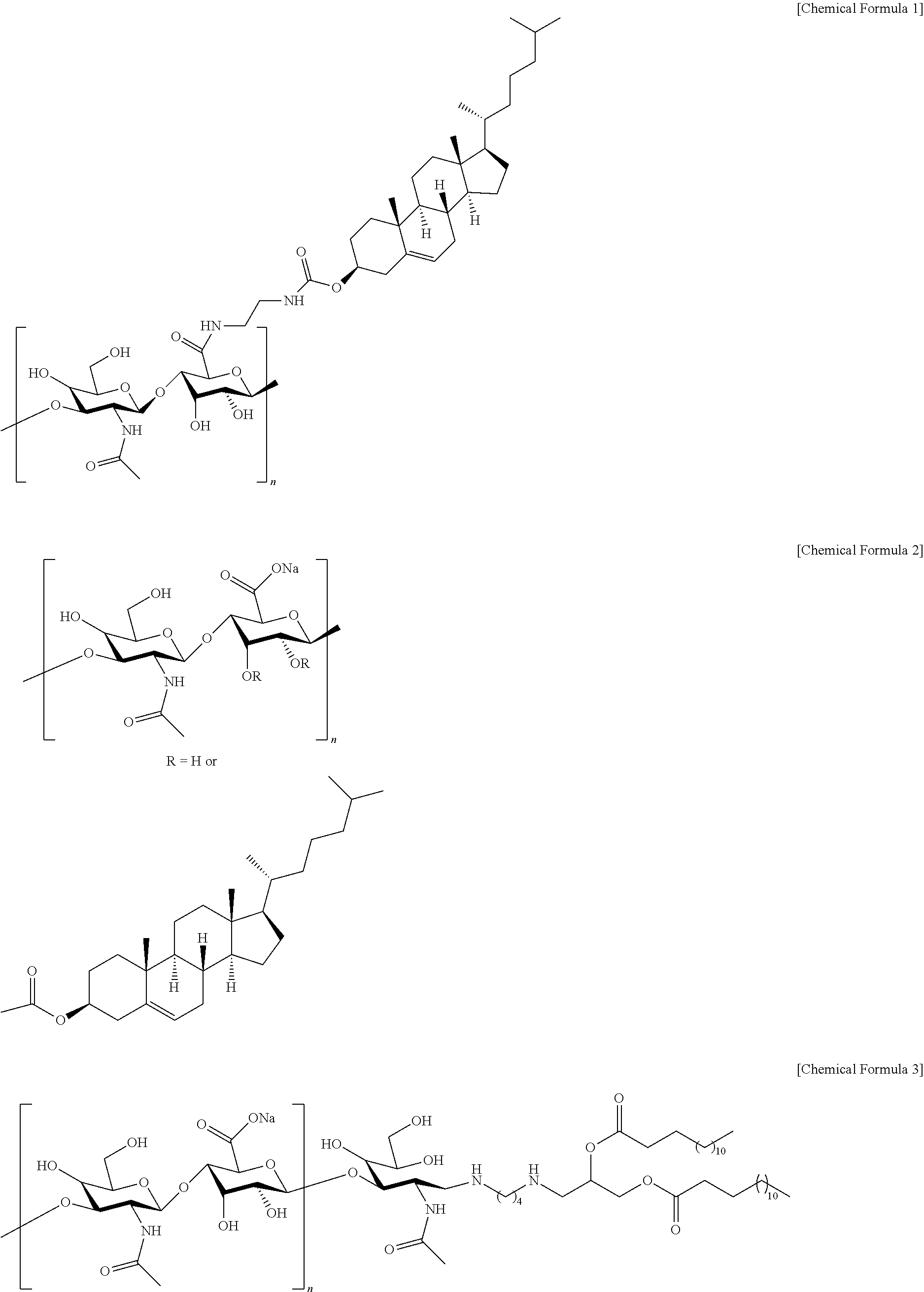

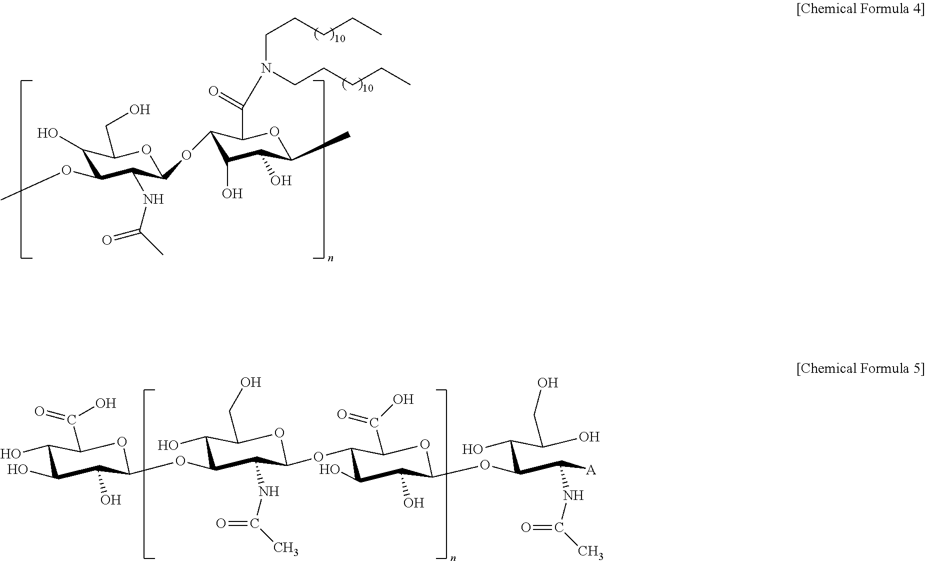

In Chemical Formulas 1 to 5, n is an integer from 1 to 50, and in Chemical Formula 5, A is an aldehyde group.

In the present invention, the compound of Chemical Formula 1 or Formula 2 may be characterized in that it is prepared by substituting the —OH group of hyaluronic acid with cholesteryl chloroformate without chemical modification.

In the present invention, the hyaluronic acid-cholesterol derivative may control the degree of cholesterol substitution of the hyaluronic acid-cholesterol derivative by adjusting the equivalent amounts of cholesteryl chloroformate, 4-dimethylaminopyridine (DMAP) and triethylamine (TEA)

In the present invention, the compound of Chemical Formula 3 may be characterized in that it is prepared by substituting the terminal of a reducing sugar of hyaluronic acid with 1,2-dimyristoyl-rac-glycerol.

In the present invention, the terminal of a reducing sugar of hyaluronic acid may be characterized in that an amine group is substituted by a ring-opening reaction.

In the present invention, the amine group may be substituted by using a diamine selected from the group consisting of ethylenediamine, butylenediamine, hexamethylenediamine, pentaethylenehexaamine, 1,4-butanediamine and 1,5-diamino-2-methylpentane, but the present invention is not limited thereto.

In terms of preparing the hyaluronic acid-lipid derivative according to the present invention, DMTMM was used as a cross-linking agent, but the cross-linking agent is not limited to DMTMM, and crosslinking agents having a similar length such as Sulfo-NHS/EDC, NHS/EDC and 2-chloro-4,6-dimethoxy-1,3,5-triazine may be substituted and used.

In terms of preparing the hyaluronic acid derivative according to the present invention, DMSO was used as a solvent, but the solvent is not limited to DMSO, and similar dipolar aprotic solvents such as N,N-dimethylmethanamide (DMF), 1,3-dimethyl-2-imidazolidinone (DMI) may be substituted and used.

The present invention provides a variety of kits that conveniently and/or efficiently carry out the methods of the present invention. Typically, a kit will include a sufficient amount and/or number of components to allow a user to multiply inoculate or treat a subject(s) and/or to perform multiple experiments.

In one aspect, the present invention provides a kit for delivering a payload of a nucleic acid, protein, compound, other material or drug, including a payload of the nucleic acid, protein, compound, other material or drug, and a lipid nanoparticle. The kit may further include packaging and instructions.

In the present invention, delivery refers to an action or method of delivering a payload such as a nucleic acid, protein, compound, other substance or drug, and in the present invention, a carrier refers to any substance that at least partially facilitates the in vivo delivery of a payload such as a nucleic acid, protein, compound, other substance or drug to a targeted cell.

### EXAMPLE

Hereinafter, the present invention will be described in more detail through examples. These examples are only provided for illustrating the present invention, and it will be apparent to those skilled in the art that the scope of the present invention is not to be construed as being limited by these examples.

### Example 1. Synthesis of Hyaluronic Acid-Lipid Derivatives

In the present invention, lipids having hydrophobic alkyl chains were introduced into hyaluronic acid to synthesize various hyaluronic acid-lipid derivatives. Sigma-Aldrich products were used for compounds and reagents that are not otherwise specified below.

### 1-1. Hyaluronic Acid-Cholesterol Derivative 1 (HA-CHOL, Chemical Formula 1)

After placing cholesteryl chloroformate (TCI) into a flask and creating a vacuum state, ethylenediamine was added, and by adding anhydrous dichloromethane (DCM, TCI) to the above flask, it was stirred for 1 hour in a<0° C. environment. Water was added to the reaction solution and washed, and all organic solvents were removed using a rotary evaporator to obtain cholesteryl-2-aminoethylcarbamate (CAEC, 1′).

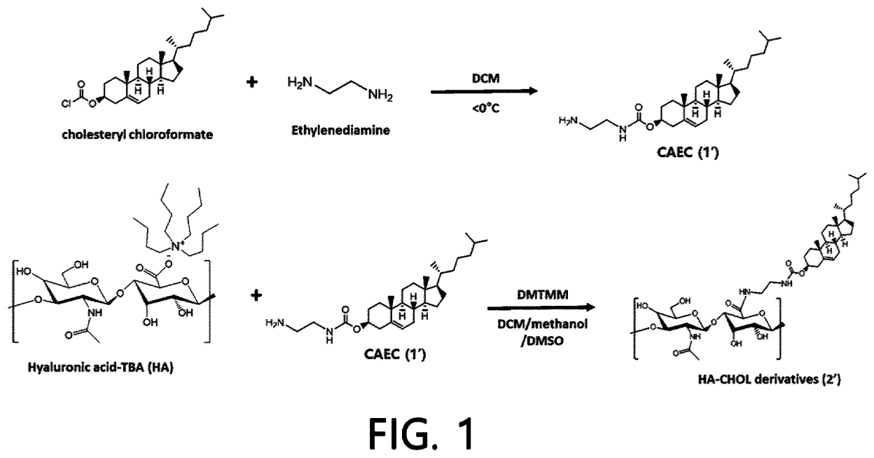

Hyaluronic acid (HA-TBA) substituted with a tetrabutylammonium (Sigma-Aldrich) salt was dissolved in DMSO. CAEC was dissolved in a DCM/methanol (1:1) solution and placed into a tetrabutylammonium salt-substituted hyaluronic acid solution, and it was stirred for 30 minutes. In the reaction solution, DMTMM (4-(4,6-dimethoxy-1,3,5-triazin-2-yl), TCI) dissolved in dimethyl sulfoxide (DMSO, Alfa Aesar) in the same molar amount as the CAEC obtained above was added, and it was stirred at room temperature for 24 hours. The prepared hyaluronic acid-cholesterol derivative 1 (HA-CHOL, 2′) was purified through dialysis and obtained through lyophilization (FIG. 1).

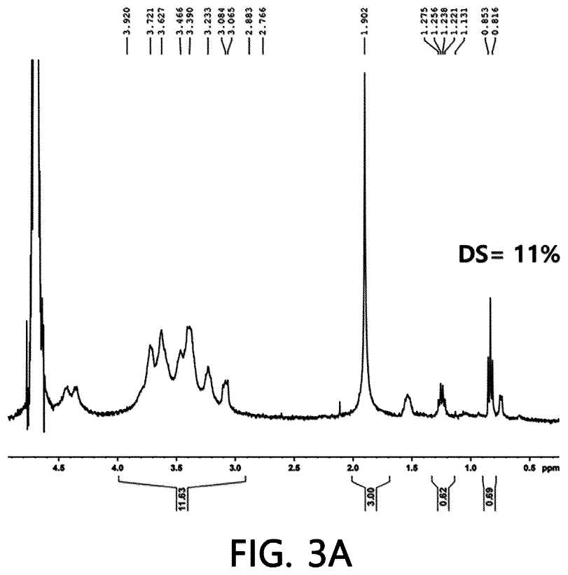

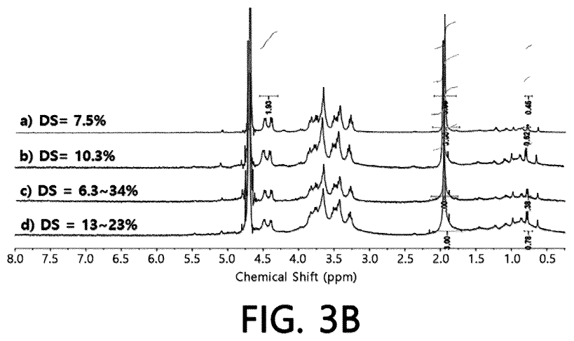

The degree of cholesterol substitution of hyaluronic acid cholesterol derivative 1 was determined through 1H NMR analysis (FIG. 3a). In the prepared hyaluronic acid-cholesterol derivative, the hydrogen integral value of the acetyl group of hyaluronic acid and the integral value of the methyl group of cholesterol are shown as 3:6, and when the integral value of the acetyl group is fixed at 3 by using the same, it is possible to calculate the degree of substitution by calculating the integral value of the methyl group.

### 1-2. Hyaluronic Acid-Cholesterol Derivative 2 (CHA, Chemical Formula 2)

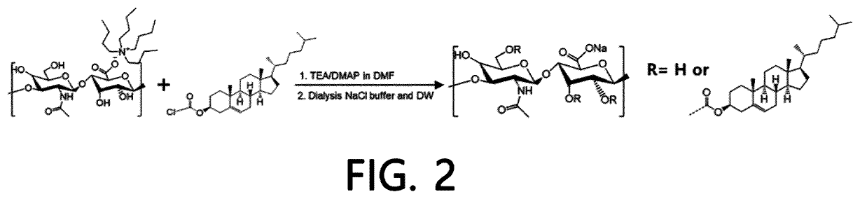

After dissolving hyaluronic acid (HA-TBA) substituted with a tetrabutylammonium salt in dimethylformamide (DMF, Alfa Aesar), cholesteryl chloroformate (TCI) and 4-dimethylaminopyridine (DMAP, TCI) were added, and triethylamine (TEA, DAEJUNG) was added and stirred. After stirring, the product was precipitated using ether to remove unreacted lipids. Afterwards, it was dialyzed in NaCl solution and distilled water for 24 hours, respectively (FIG. 2).

The degree of cholesterol substitution of hyaluronic acid cholesterol derivative 2 was determined through 1H NMR analysis.

Meanwhile, in the case of hyaluronic acid cholesterol derivative 2, the degree of cholesterol substitution may be adjusted according to the reaction composition (Table 1). That is, the hydrogen integral value of the acetyl group of hyaluronic acid and the integral value of the methyl group of cholesterol of the prepared hyaluronic acid-cholesterol derivative are shown as 3:6, and when the integral value of the acetyl group is fixed to 3 by using the same, it is possible to calculate the degree of substitution by calculating the integral value of the methyl group. As the substitution degree of cholesterol increases, the hydrogen integral of the acetyl group of hyaluronic acid overlaps with the hydrogen integral of cholesterol, and thus, the degree of substitution of cholesterol may be calculated based on the integral value of hydrogen at the l′ carbon of the glycidic acid bond of hyaluronic acid as 2 (FIG. 3b). The cholesterol-substituted hyaluronic acid derivative was named CHA.

TABLE 1
Composition information of
hyaluronic acid-cholesterol derivative 2

| Entry | HA | Cholesterol | DMAP | TEA |
|---|---|---|---|---|
| a) | 450 mg | 98 mg | 67 mg | 111 mg |
| b) | 450 mg | 197 mg | 134 mg | 111 mg |
| c) | 450 mg | 296 mg | 134 mg | 111 mg |
| d) | 450 mg | 296 mg | 134 mg | 111 mg |

### 1-3. Hyaluronic Acid-1,2-Dimyristoyl-Rac-Glycerol Derivative (DMG-HA, Chemical Formula 3)

As the hyaluronic acid, 4K low-molecular-weight hyaluronic acid prepared by the inventors of the present invention was used.

For the 4K low-molecular-weight hyaluronic acid, high-molecular-weight hyaluronic acid (sodium hylauronate (1.0 to 1.5 mDA), Biogenics) was dispersed in acetone containing less than 5% of salt in water, and an aqueous solution of hydrochloric acid (SAMCHUN) (3M) was additionally added at high temperature (60 to 70° C.) and stirred for 24 hours, and then, hyaluronic acid was precipitated and filtered. Thereafter, the filtered hyaluronic acid was recovered and obtained by washing with an ethanol solution (Ethanol, 95%, SAMCHUN).

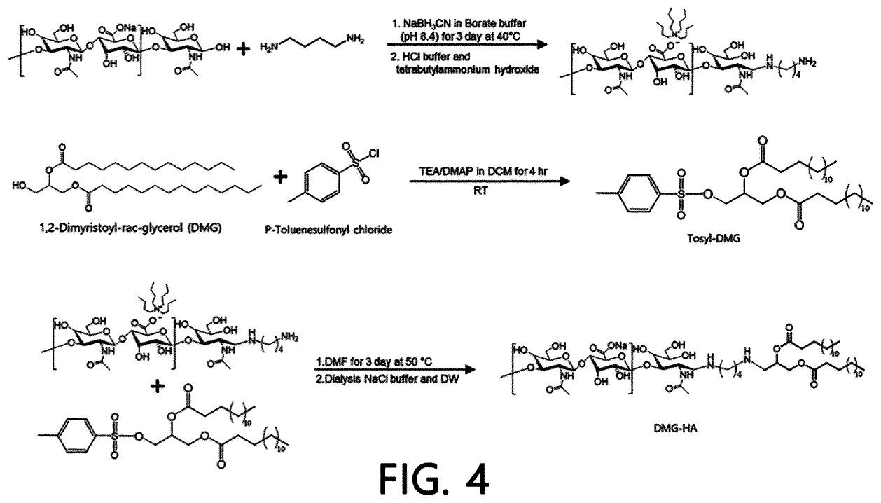

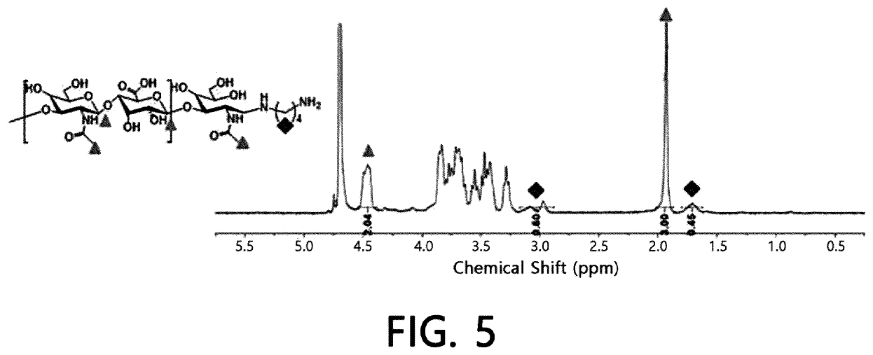

1 g of hyaluronic acid was dissolved in 100 mL of sodium borate buffer (pH 8.5). By using a pH meter, it was confirmed that the pH was not lowered to 8.4 or less. When the pH was less than 8.4, it was titrated to pH 8.4 using 1M NaOH solution. Thereafter, a diamine compound, in this case, diaminobutane (Sigma Aldrich), was added to the above solution in an amount of 15 times compared to 2 equivalent amounts of glucose in hyaluronic acid. After sufficiently stirring, sodium cyanoborohydride (Sigma Aldrich) was additionally added to the solution in an equivalent amount of 5 times compared to 2 equivalent amounts of glucose in hyaluronic acid, and the mixture was stirred at 40° C. for 3 days. The above reaction solution was concentrated to a volume of 20 mL through a vacuum evaporator, placed into a dialysis membrane having MWCO 3500 and dialyzed with distilled water for 1 day. After additional dialysis with 0.1M HCl solution for 1 day, finally, dialysis was performed with tertiary distilled water for 1 day. The product obtained through this is a hyaluronic acid-b-diaminobutane derivative, in which sodium of hyaluronic acid is removed. This was named HA-b-damb. 40% tetrabutylammonium hydroxide (TBA-OH, Sigma Aldrich) was added to the resulting product solution to adjust the pH to 7.0. Due to TBA-OH, HA-b-damb was substituted with a TBA salt, and this was named HA-damb-TBA. Afterwards, the product was obtained through lyophilization. The synthesis of HA-b-damb and HA-b-damb-TBA was confirmed by 1H NMR analysis. It was confirmed that all of the sugar unit of hyaluronic acid, the unique methylene hydrogen of diaminobutane and the alkyl chain of TBA salt appeared. When comparing the integral value of diaminobutane with the integral value of the acetyl group of hyaluronic acid, it was confirmed that about 1 diaminobutane was substituted for every 10 hyaluronic acid sugar units, and it was found that diaminobutane was substituted at all 4k hyaluronic acid terminals (FIGS. 5 and 6).

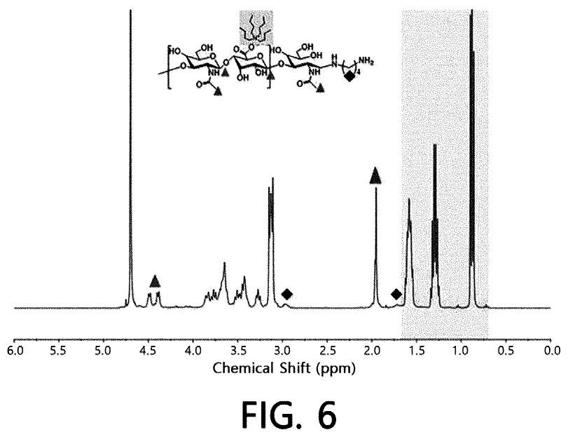

0.25 mmol of 1,2-dimyristoyl-rac-glycerol (DMG, GOLDBIO), 0.3 mmol of P-toluenesulfonyl chloride (TsCl, SAMCHUN) and 0.125 mmol of 4-dimethylaminopyridine (DMAP) were dissolved together in 1.2 mL of dichloromethane (DCM, TCI), and anhydrous state was maintained through nitrogen gas. Afterwards, 0.3 mmol of triethylamine (TEA, DAEJUNG) was added using a syringe, and the mixture was sufficiently stirred at room temperature for 4 hours. The product was confirmed by TLC (TLC conditions:mobile solvent DCM; RF value 0.52). The reactants were recovered, and only the product was recovered using silica gel chromatography. The recovered product was recovered through dark concentrated distilled water. This was named Tosyl-DMG. The synthesis of Tosyl-DMG was confirmed through 1H NMR analysis. The integral values of hydrogen in the unique alkyl chain and tosyl functional group of DMG were consistent with the chemical formula, indicating that the synthesis was successful (FIG. 7).

The previously prepared HA-damb-TBA and Tosy-DMG were dissolved in anhydrous DMF. The mixture was stirred using a magnetic stirrer for 72 hours at 50° C. to 60° C. while maintaining an anhydrous state using nitrogen gas. After completion of the reaction, the reaction solution was recovered, and ether corresponding to 10 times the volume of the reaction solution was added to precipitate the product, which was recovered through centrifugation. This can also be recovered using a filter. Once again, ether (SAMCHUN) corresponding to 10 times the volume of the product was added to the precipitate to dissolve, and it was centrifuged to remove all unreacted Tosyl-DMG. The precipitated product was dissolved in water, and after dialyzing using a 0.2 M NaCl solution for 24 hours, dialysis was performed using distilled water for additional 24 hours. Afterwards, the product was obtained through lyophilization. The hyaluronic acid-lipid derivative thus obtained was named DMG-HA.

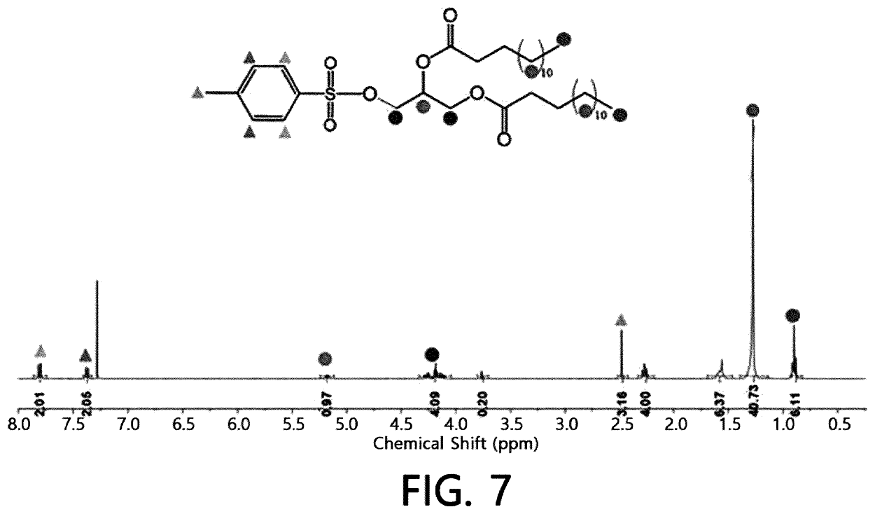

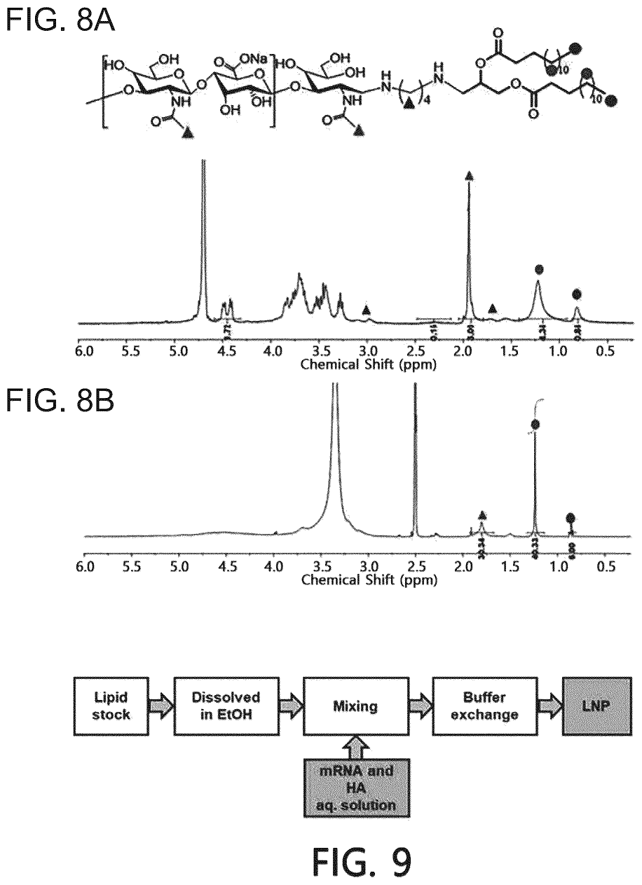

The synthesis of DMG-HA was confirmed through 1H NMR analysis. As a result of dissolving DMG-HA in D₂O and measuring, hydrogen peaks appearing in the unique sugar units of hyaluronic acid and in the alkyl chain of DMG were confirmed. In the results measured by dissolving in DMSO-d6, the hydrogen integral value of the alkyl chain of DMG in DMG-HA was consistent with the hydrogen integral value of DMG before substitution, and accordingly, it was found that DMG was not decomposed but was substituted at the terminal of hyaluronic acid. In addition, when it was compared based on the acetyl group of hyaluronic acid, it was confirmed that DMG was substituted at all hyaluronic acid terminals (FIG. 8).

### 1-4. Synthesis of Hyaluronic Acid-Ditetradecyalacetamide (Chemical Formula 4)

Similar to the synthesis method of Example 1-1, hyaluronic acid-Ditetradecylacetamide may also be prepared. In this case, ditetradecylamine (dimethyltetradecylamine, Sigma-Aldrich) is used instead of cholesteryl chloroformate (TCI). Hyaluronic acid (HA-TBA) substituted with a tetrabutylammonium (tetrabutylammonium, Sigma-Aldrich) salt was dissolved in DMSO. Ditetradecylamine was dissolved in a DCM/methanol (1:1) solution, and it was added to the hyaluronic acid solution and stirred for 30 minutes. Dissolved DMTMM which was dissolved in dimethyl sulfoxide (DMSO, Alfa Aesar) in the same molar number as the CAEC obtained above was added to the reaction solution and stirred at room temperature for 24 hours. The prepared hyaluronic acid-dietetradecylacetamide derivative (4′, HA-DAT) was purified through dialysis and obtained through lyophilization.

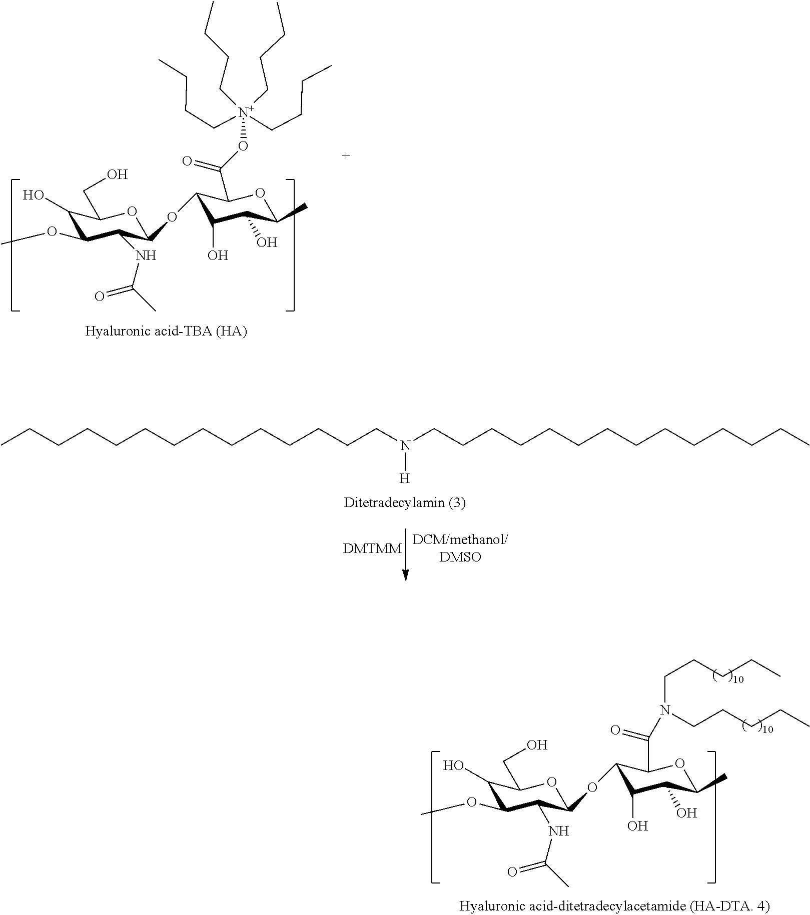

### 1-5. Synthesis of Hyaluronic Acid-Lipid Derivative (Chemical Formula 5) Using Hyaluronic Acid Aldehyde

A of hyaluronic acid aldehyde includes an aldehyde group, and A is an aldehyde group. It is possible to synthesize a hyaluronic acid-lipid derivative using a method similar to the example by using an aldehyde group.

Specifically, hyaluronic acid was dissolved in pH 8.5 sodium borate buffer (borate salt buffer solution) to prepare a hyaluronic acid solution having a hyaluronic acid concentration of 10 mg/mL, and sodium cyanoborohydride, which is a reducing agent, was added to the solution in an amount of 5 mole times of the aldehyde group in hyaluronic acid. Thereafter, diaminobutane (1,4-diaminobutane, Sigma Aldrich) was added in an amount of 10 mole times of the aldehyde group and reacted at 37° C. for 3 days to prepare a hyaluronic acid-diaminobutane derivative. The prepared hyaluronic acid-diaminobutane derivative was dialyzed in distilled water for 3 days using a dialysis tubing (7000K MWCO, Snakeskin), lyophilized and then dissolved again in a pH 8.5 borate salt buffer solution to prepare a solution in which the concentration of the derivative was 10 mg/mL. To this solution, sodium cyanoborohydride, which is a reducing agent, was added in an amount of 10 mole times of the amine group. Afterwards, glutaraldehyde (glutaraldehyde solution, Sigma Aldrich) was added in an amount of 10 mole times of the amine group and reacted at room temperature for 1 day. The prepared hyaluronic acid-glutaraldehyde derivative was dialyzed against distilled water for 3 days using a dialysis tubing (7000K MWCO, Snakeskin) and lyophilized for 3 days. A solution was prepared by dissolving the prepared hyaluronic acid-glutaraldehyde derivative in a pH 5.5 sodium acetate buffer solution. Sodium cyanoborohydride, which is a reducing agent, was added in an amount of 10 mole times of the aldehyde group of the derivative added to the solution. Thereafter, the hyaluronic acid-glutaraldehyde derivative was reacted at a molar ratio of 1 to 5:1 to synthesize a hyaluronic acid-lipid derivative using hyaluronic acid aldehyde.

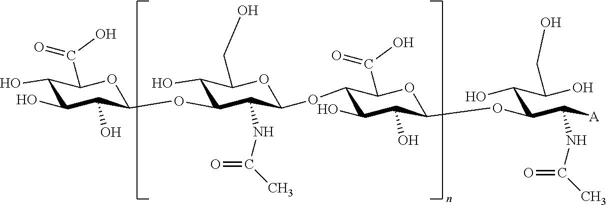

In the method of synthesizing the hyaluronic acid-lipid derivative, the cross-linking agent is not limited to DMTMM, and similar crosslinking agents such as Sulfo-NHS/EDC, NHS/EDC or 2-chloro-4,6-dimethoxy-1,3,5-triazine may be used.

In the method of synthesizing the hyaluronic acid-lipid derivative, the solvent is not limited to DMSO, and dipolar aprotic solvents such as N,N-dimethylmethanamide (dimethylformamide, DMF) or 1,3-dimethyl-2-imidazolidinone (DMI) may be used.

### Example 2. Preparation of Nanoparticles Including Hyaluronic Acid-Lipid Derivatives

A cationic lipid (SM-102, CAYMAN CHEMICAL COMPANY), phosphatidylcholine (DSPC, Avanti Polar Lipids), cholesterol (Cholesterol, Sigma Aldrich) and the hyaluronic acid-lipid derivative were prepared in by dissolving in ethanol (Sigma Aldrich) with a purity of 90% or more. In this case, it was dissolved by adding 40% or more of the cationic lipid, 10% or more of phosphatidylcholine, 30% or more of cholesterol, and 0.1% or more of hyaluronic acid-lipid derivative based on the total volume of the lipid mixture.

A nucleic acid was dissolved in 10 mM citrate buffer. In this case, the hyaluronic acid-lipid derivative may also be mixed together depending on cases.

In the case of using a microfluidic mixing device, the lipid mixture and the citrate buffer including a supporting material were mixed by flowing through a syringe at a rate of at least 0.3 mL/min at a ratio of 1:3, respectively. Thereafter, the mixture was quickly placed into a phosphate buffer such that the concentration of the organic solvent in the total solution volume was less than 5%.

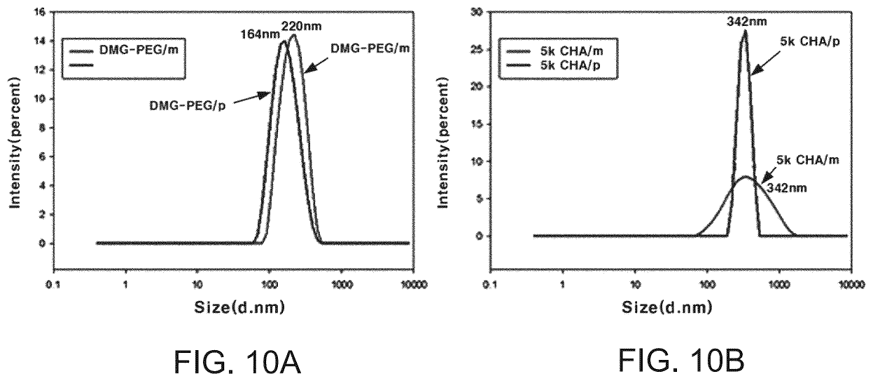

In the case of preparation by pipetting, pipetting was performed more than 20 times to mix well. Alcohol was removed by using a 3.5 kD to 100 kD filtration membrane or a centrifugal filter, and buffer exchange was performed. In the case of using a filtration membrane, alcohol was completely removed by exposing the filtration membrane to a phosphate buffer for 24 hours or more, and in the case of using a centrifugal filter, alcohol was completely removed by exchanging the membrane with phosphate buffer at 1,000 g for 3 minutes or more twice or more. (FIG. 10).

The results of comparing the sizes of the microfluidic mixing device and the LNP prepared in vitro are shown in FIG. 10. The size of the LNP was measured by a dynamic light scattering spectrometer (ZEN3600, Malvern Instruments) after dispersing the prepared LNP in a phosphate buffer.

It was confirmed that lipid nanoparticles were synthesized in both cases of using a microfluidic mixing device or pipetting.

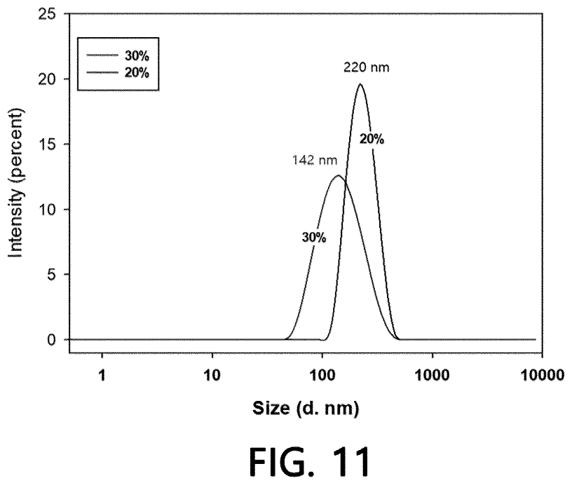

As some embodiments used in the present invention, LNPs including hyaluronic acid-lipids were prepared with the compositions shown in Table 2 or Table 3. In Table 3, as a large amount of hyaluronic acid-lipid derivative was added, the aggregation of nanoparticles could be suppressed (FIG. 11).

TABLE 2
LNP composition including hyaluronic acid-lipid

| | | | Lipid | |
|---|---|---|---|---|
| | | | com- | Lipid |
| | | | position | concen- |
| | | | molar | tration: |
| LNPs | Lipid | Mw | ratio | buffer |
| DMG-PEG/ | SM-102 | 710.2 | 49 | 1:3 |
| 4k CHA | DSPC | 790.2 | 10 | |
| | Cholesterol | 386.654 | 39 | |
| | DMG-PEG | 2509.2 | 1 | |
| | 4k CHA | 4386.65 | 1 | |
| 4k CHA | SM-102 | 710.2 | 50 | |
| | DSPC | 790.2 | 10 | |
| | Cholesterol | 386.654 | 39 | |
| | 4k CHA | 4386.65 | 1 | |
| DMG-HA | SM-102 | 710.2 | 48 | |
| x5 | DSPC | 790.2 | 9 | |
| | Cholesterol | 386.654 | 38 | |
| | DMG-HA | 5000 | 5 | |
| DMG-PEG | SM-102 | 710.2 | 50 | |
| (Control) | DSPC | 790.2 | 10 | |
| | Cholesterol | 386.654 | 39 | |
| | DMG-PEG | 2509.2 | 1 | |

*Herein, 4K is the molecular weight information of hyaluronic acid. 4K hyaluronic acid was prepared in the manner described in Examples 1-3.

TABLE 3
LNP composition including hyaluronic acid-lipid

| | | | Lipid | Lipid |
|---|---|---|---|---|
| | | | com- | concen- |
| | | | position | tration: |
| LNPs | Lipid | Mw | molar ratio | buffer |
| 5k CHA | SM-102 | 710.2 | 46 | 1:3 |
| 50% | DSPC | 790.2 | 9 | |
| | Cholesterol | 386.654 | 35 | |
| | 5k CHA | 5386.65 | 10 | |
| 5k CHA | SM-102 | 710.2 | 47 | |
| 40% | DSPC | 790.2 | 9 | |
| | Cholesterol | 386.654 | 37 | |
| | 5k CHA | 5386.65 | 7 | |
| 5k CHA | SM-102 | 710.2 | 48 | |
| 30% | DSPC | 790.2 | 10 | |
| | Cholesterol | 386.654 | 38 | |
| | 5k CHA | 5386.65 | 4 | |
| 5k CHA | SM-102 | 710.2 | 49 | |
| 20% | DSPC | 790.2 | 10 | |
| | Cholesterol | 386.654 | 38 | |
| | 5k CHA | 5386.65 | 3 | |

*Herein, 5K is the molecular weight information of hyaluronic acid (Sodium Hyaluronate, Research Grade, HA5K, Lifecore Biomedical).

### Example 3. Comparison of LNP Sizes According to the Amount of DMG-HA

LNPs including DMG-HA were prepared according to the compositions shown in Table 4.

TABLE 4
LNP composition including hyaluronic acid-lipid

| | DMG-HA x1 | DMG-HA x5 | DMG-HA x10 |
|---|---|---|---|
| Lipids | (molar ratio) | (molar ratio) | (molar ratio) |
| SM-102 | 50 | 48 | 46 |
| DSPC | 10 | 10 | 9 |
| Cholesterol | 39 | 37 | 35 |
| DMG-HA | 1 | 5 | 10 |

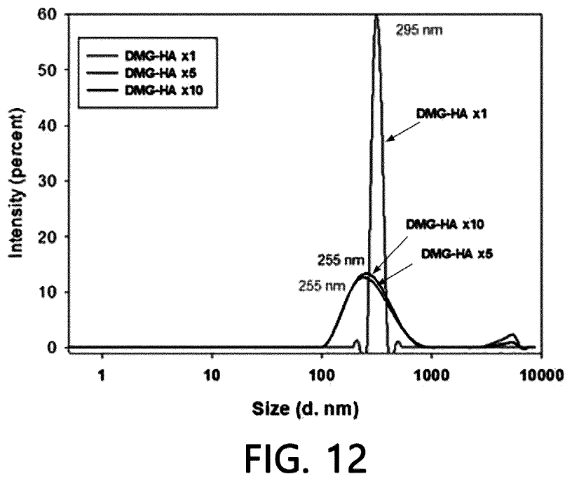

As a result, it was confirmed that nanoparticles were most uniformly formed in the case of DMG-HA x1. (FIG. 12).

### Example 4. Comparison of the Sizes of LNPs Including CHA and DMG-PEG

LNPs including CHA and DMG-PEG was prepared with the compositions shown in Table 5.

TABLE 5
LNP composition including CHA and DMG-PEG

| | | DMG-PEG/ | |
|---|---|---|---|
| | DMG-PEG | 4K CHA | 4K CHA |
| Lipids | (molar ratio) | (molar ratio) | (molar ratio) |
| SM-102 | 50 | 49 | 50 |
| DSPC | 10 | 10 | 10 |
| Cholesterol | 39 | 39 | 39 |
| DMG-PEG | 1 | 1 | — |
| 4K CHA | — | 1 | 1 |

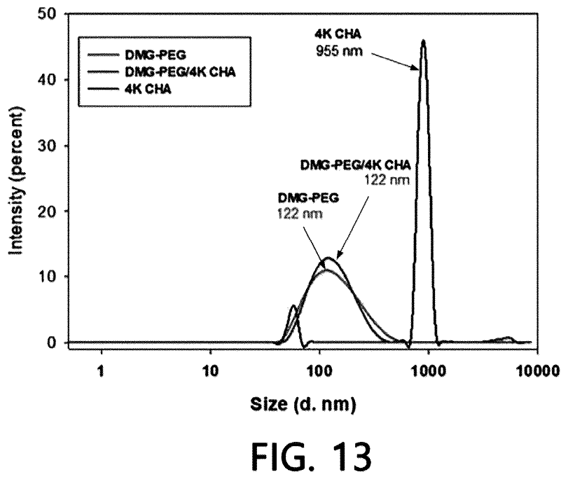

As a result, it was confirmed that when LNP was prepared by adding CHA alone, the size was large but the dispersion was small, indicating a uniform size, whereas it was found that when LNP was prepared by adding CHA and DMG-PEG together, the nanoparticles were generated somewhat non-uniformly (FIG. 13).

### Example 6. Zeta Potential Analysis of LNPs Including Hyaluronic Acid-Lipid Derivatives

LNPs including hyaluronic acid-lipid derivatives were prepared with the composition according to Table 2, and zeta potential was analyzed. The zeta potential was measured by a dynamic light scattering spectrometer (ZEN3600, Malvern Instruments) after dispersing the prepared LNP in a phosphate buffer.

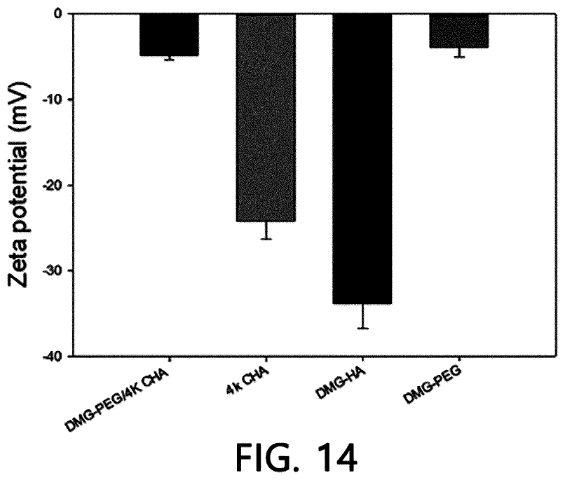

As a result, when CHA and DMG-HA were added, the negative value of the zeta potential of LNP was shown to be larger than when DMG-PEG was added or DMG-PEG and CHA were mixed and added, and it was confirmed that the hyaluronic acid lipid according to the present invention was included more effectively in the LNP (FIG. 14).

### Example 7. Comparison of the Sizes and Zeta Potentials of LNPs Including a Combination of CHA and DMG-PEG or DMG-HA and DMG-PEG

When lipid nanoparticles were prepared by adding a PEG derivative and a hyaluronic acid derivative together, LNPs including a combination of CHA and DMG-PEG or DMG-HA and DMG-PEG were prepared in the compositions shown in Table 6 in order to confirm the appropriate composition ratio of the same.

TABLE 6
LNP composition including a combination of CHA
and DMG-PEG or DMG-HA and DMG-PEG

| | | DMG-PEG/ | DMG-PEG/ |
|---|---|---|---|
| | DMG-PEG | 4K CHA | DMG-HA |
| Lipids | (molar ratio) | (molar ratio) | (molar ratio) |
| SM-102 | 50 | 50 | 50 |
| DSPC | 10 | 10 | 10 |
| Cholesterol | 39 | 39 | 39 |
| DMG-PEG | 1 | 1 | 1 |
| 4K CHA | — | 0.1 | — |
| DMG-HA | — | — | 0.1 |

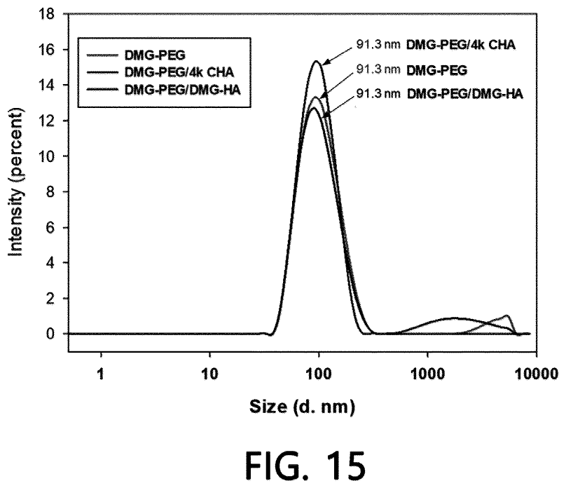

As a result, it was confirmed that the LNPs including only DMG-PEG, which were DMG-PEG/4K CHA, DMG-PEG/DMG-HA LNP, showed a uniform size with low dispersion (FIG. 15).

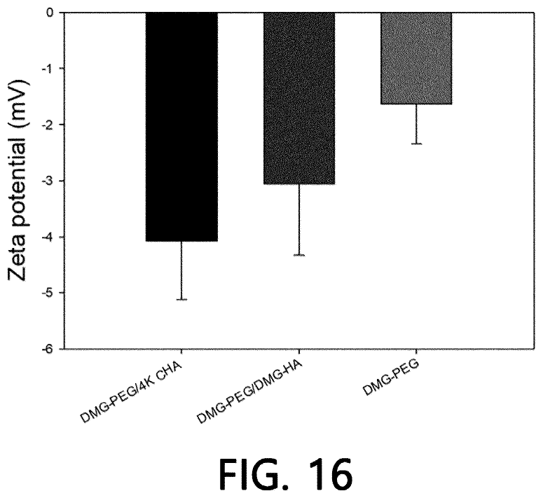

Meanwhile, as a result of preparing LNPs with the compositions according to Table 6 and analyzing the zeta potential, the DMG-PEG/4K CHA, DMG-PEG/DMG-HA LNP showed a negative value of zeta potential of the LNPs compared to the LNPs including only DMG-PEG, and it was confirmed that when PEG derivatives and hyaluronic acid derivatives are added together at an appropriate ratio, hyaluronic acid lipids are effectively included in LNPs (FIG. 16).

### Example 8. Evaluation of Delivery Efficiency into mRNA Cells of LNPs Including Hyaluronic Acid-Lipid Derivatives

In Table 2, LNPs including DMG-PEG/4K CHA, DMG-HA and DMG-PEG were respectively prepared, and GFP mRNA (Clean Cap®EGFP mRNA, TriLink) was loaded thereon.

LNP preparation was prepared by dispersing each lipid composition in an organic solvent, dissolving mRNA and hyaluronic acid lipid in citrate buffer, and mixing by pipetting at a ratio of 1:3. The mRNA and hyaluronic acid lipids were prepared by mixing DMG-PEG/4K CHA at a ratio of 1:1.6, DMG-HA at a ratio of 1:9.3, and DMG-PEG at a ratio of 1:0.9 based on mass in citrate buffer.

A549 cells (Korea Cell Line Bank) were dispensed at 1×10³ cells/well in a 96-well plate, and then cultured overnight in a CO₂ incubator at 37° C. using a culture medium (RPMI medium 1640 (1X), Gibco). Each LNP loaded with GFP mRNA was diluted in a culture medium containing 5% FBS (RPMI medium 1640 (1X), Gibco), and each well was treated with 100 ng of mRNA standard. The LNP-treated cells were additionally cultured for 24 hours in a CO₂ incubator at 37° C., and then, GFP expression was confirmed through a fluorescence microscope.

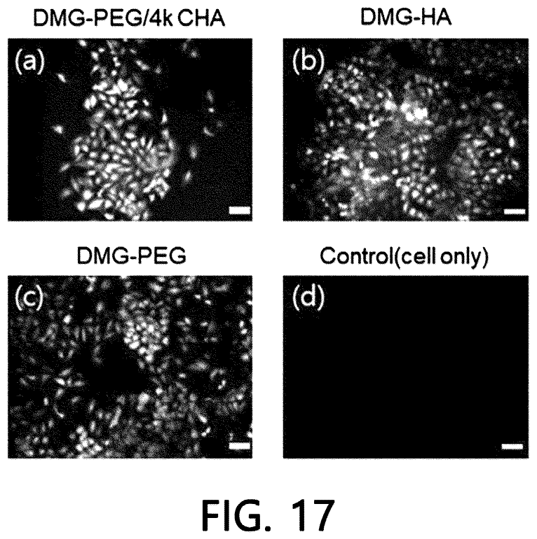

As a result, it was confirmed that the GFP mRNA was most effectively delivered into cells by the LNP including DMG-HA alone compared to the LNP including DMG-PEG alone or a mixture of DMG-PEG and CHA such that the GFP expression level was shown to be the highest (FIG. 17).

Meanwhile, after preparing the LNPs with the compositions according to Table 6, GFP mRNA was loaded and treated in cells in the same manner as above, and the intracellular delivery efficiency was evaluated. The mRNA and hyaluronic acid lipids were prepared by mixing DMG-PEG/4K CHA at a ratio of 1:0.16, DMG-PEG/DMG-HA at a ratio of 1:0.15, and DMG-PEG at a ratio of 1:0.9 based on mass in citrate buffer. Lipofectamine was prepared according to the manufacturer's manual such that 100 ng of mRNA and 0.15 μL of a lipofectamine reagent were added per. In this case, imaging was performed by utilizing IVIS (In vivo imaging system, Perkin Elmer), and fluorescence was quantitatively analyzed.

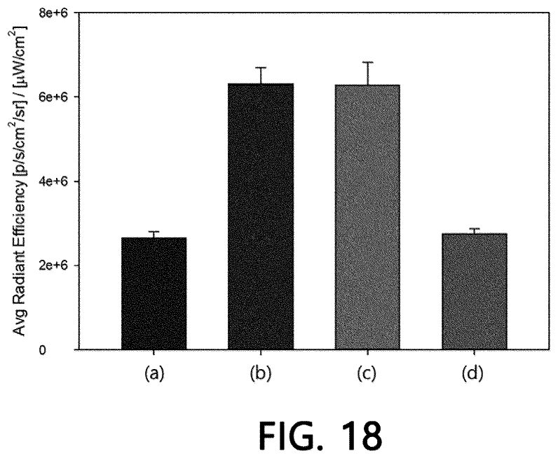

As a result, it was confirmed that when the PEG derivative and the hyaluronic acid derivative were added together at an appropriate ratio, the mRNA was effectively loaded onto the LNP (FIG. 18).

### Example 9. Evaluation of mRNA Loading Efficiency of LNPs Including Hyaluronic Acid-Lipid Derivatives

The mRNA loading efficiency was evaluated by varying the amount of 5K CHA in the compositions according to Table 3. To this end, in order to measure the mRNA in the buffers inside and outside the LNPs, the Ribogreen dye solutions with and without TritonX-100 (Sigma-Aldrich) were treated, respectively, and it was confirmed through a plate fluorescence reader at an excitation wavelength of 480 nm and an emission wavelength of 520 nm of the Ribogreen assay. Specifically, the experiment was conducted according to the manufacturer's method by using the Ribogreen assay Kit (Quant-it™ Ribogreen RNA Assay Kit, Invitrogen™).

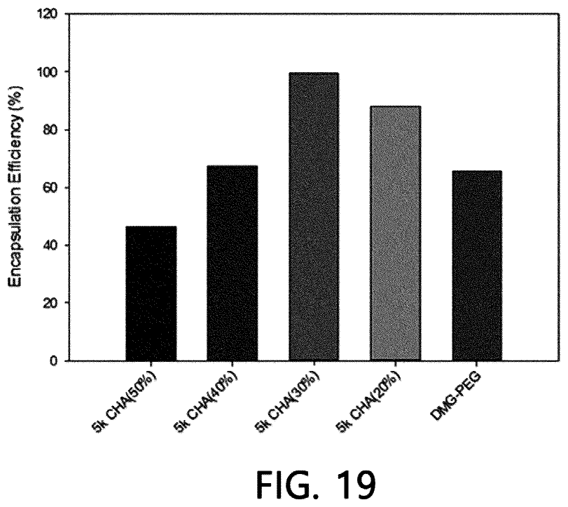

As a result, the mRNA loading efficiency of LNPs including 20 to 30% CHA was improved by more than 20% than that using DMG-PEG, and particularly, in the case of LNPs including 30% CHA, it was confirmed that the mRNA loading efficiency was close to 100% (FIG. 19).

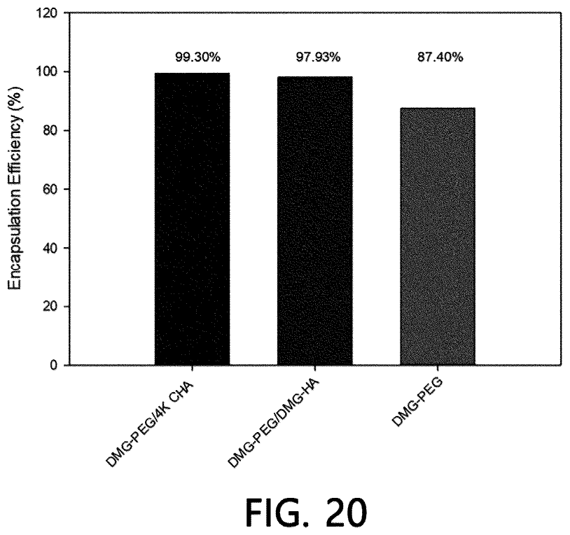

Meanwhile, as a result of evaluating the loading efficiency of the compositions according to Table 6, in the LNP prepared by mixing DMG-HA and 4k CHA with DMG-PEG at an appropriate ratio, the mRNA loading efficiency was improved by about 10% or more, compared to the LNP including DMG-PEG without a hyaluronic acid derivative (FIG. 20).

Having described specific parts of the content of the present invention in detail above, it will be clear to those skilled in the art that these specific descriptions are only preferred exemplary embodiments, and the scope of the present invention is not limited thereto. Accordingly, the substantial scope of the present invention will be defined by the appended claims and their equivalents.

1. A lipid nanoparticle, comprising a hyaluronic acid-lipid derivative.

2. The lipid nanoparticle of claim 1, further comprising an ionic lipid, cholesterol and phosphatidylcholine.

3. The lipid nanoparticle of claim 1, wherein the ionic lipid is a cationic lipid.

4. The lipid nanoparticle of claim 1, wherein the hyaluronic acid-lipid derivative is formed by introducing a lipid into the —COOH group or —OH group of a hyaluronic acid sugar skeleton or a reducing sugar at the terminal of hyaluronic acid.

5. The lipid nanoparticle of claim 4, wherein in the hyaluronic acid-lipid derivative, the lipid is a lipid having a hydrophobic alkyl chain.

6. The lipid nanoparticle of claim 1, wherein the hyaluronic acid-lipid derivative is a hyaluronic acid-cholesterol derivative or hyaluronic acid-1,2-dimyristoyl-rac-glycerol.

7. The lipid nanoparticle of claim 1, wherein the lipid nanoparticle further comprises lapidated PEG.

8. The lipid nanoparticle of claim 1, wherein the lipid nanoparticle is for delivering a nucleic acid, a protein or a drug.

9. The lipid nanoparticle of claim 8, wherein the nucleic acid is mRNA.

10. The lipid nanoparticle of claim 1, wherein the molecular weight of the hyaluronic acid is 3,000 to 6,000 Da.

11. The lipid nanoparticle of claim 1, wherein the molar ratio of ionic lipid:cholesterol:phosphatidylcholine:hyaluronic acid-lipid derivative in the lipid nanoparticle is 30 to 60:30 to 60:8 to 12:1 to 10.

12. The lipid nanoparticle of claim 1, wherein the lipid nanoparticle is administered by a route selected from the group consisting of intravenous, intramuscular, intravitreal, intrathecal, intratumoral, intranasal, pulmonary and subcutaneous routes.

13. A vaccine composition, comprising an mRNA vaccine and the lipid nanoparticle according to claim 1.

14. A hyaluronic acid-lipid derivative, which is represented by any one of Chemical Formulas 1 to 4 below: 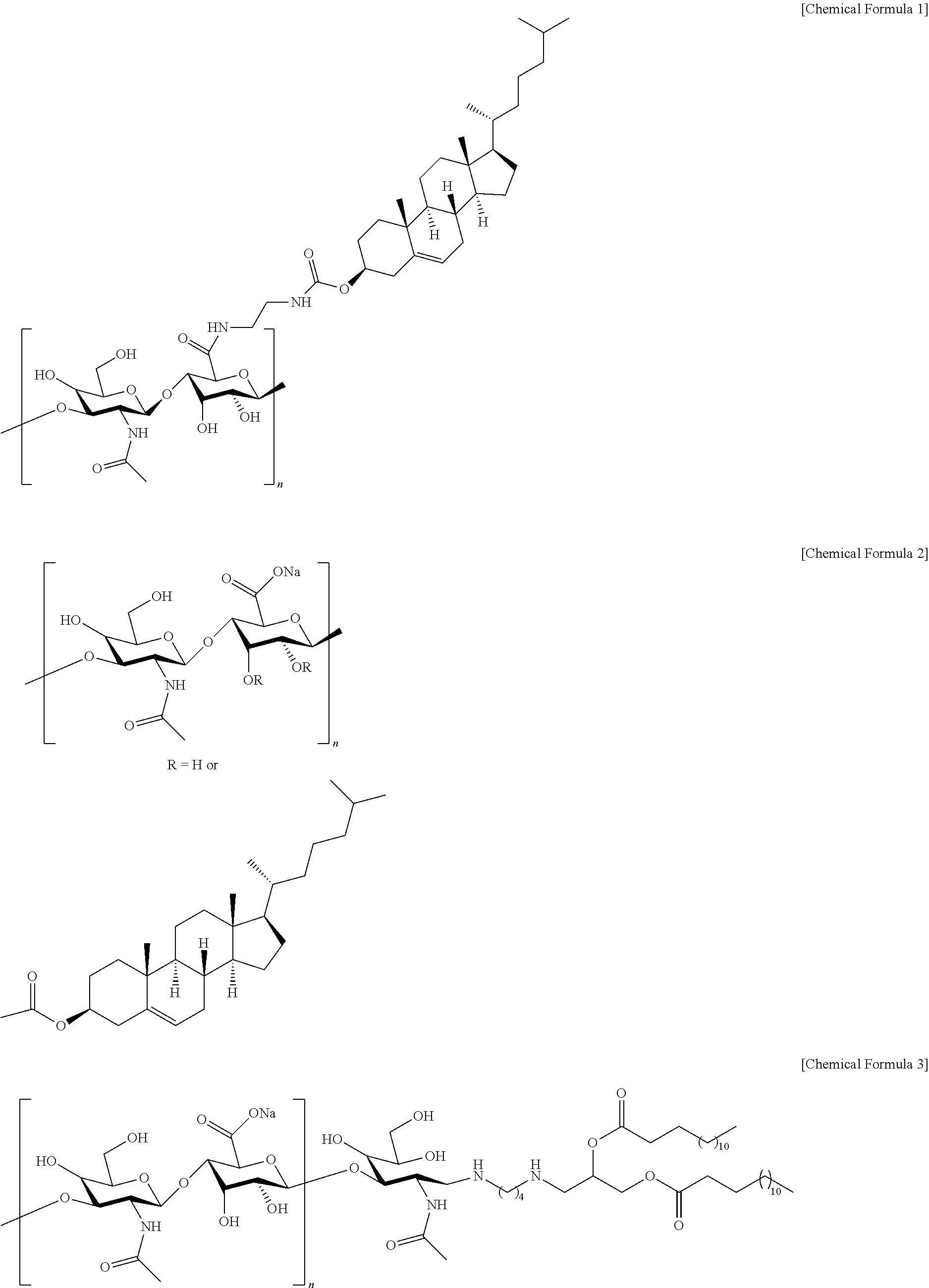 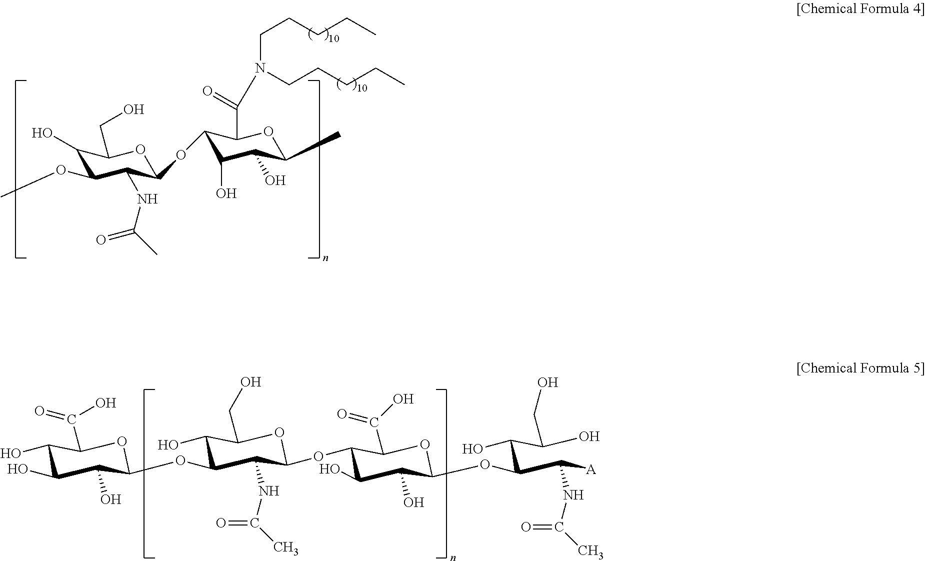
wherein in Chemical Formulas 1 to 5, n is an integer from 1 to 50, and in Chemical Formula 5, A is an aldehyde group.

15. The hyaluronic acid-lipid derivative of claim 14, wherein the compound of Chemical Formula 1 or Chemical Formula 2 is prepared by substituting the —OH group of hyaluronic acid with cholesteryl chloroformate without chemical modification.

16. The hyaluronic acid-lipid derivative of claim 15, wherein the hyaluronic acid-lipid derivative controls the degree of cholesterol substitution of the hyaluronic acid-cholesterol derivative by adjusting the equivalent amounts of cholesteryl chloroformate, 4-dimethylaminopyridine (DMAP) and triethylamine (TEA).

17. The hyaluronic acid-lipid derivative of claim 14, wherein the compound of Chemical Formula 3 is prepared by substituting the terminal of a hyaluronic acid reducing sugar with 1,2-dimyristoyl-rac-glycerol.

18. The hyaluronic acid-lipid derivative of claim 17, wherein the terminal of the hyaluronic acid reducing sugar is substituted with an amine group through a ring-opening reaction.

19. The hyaluronic acid-lipid derivative of claim 18, wherein the amine group is substituted by using a diamine selected from the group consisting of ethylenediamine, butylenediamine, hexamethylenediamine, pentaethylenehexamine, 1,4-butanediamine and 1,5-diamino-2-methylpentane.

20. A method for delivering a vaccine or medicine to a subject in need thereof comprising administering the lipid nanoparticle of claim 1 comprising the vaccine or medicine.
